SEQUENCE LISTING (6 sequences)

SEQ ID NO: 1 - protein, 180 aa, Leishmania infantum
ELATEWEDALRERALAERDEAAAAELDAAASTSQNARESACERLTSLEQQLRDSEERAAELASQLESTTAAKMSAEQDRESTRATLEQQLRESEERAAELASQLESTTAAKMSAEQDRESTRATLEQQLRDSEERAAELASQLESTTAAKMSAEQDRESTRATLEQQLRESEARAAELAS

SEQ ID NO: 2 - protein, 879 aa, Trypanosoma cruzi
MGRTVVGASRMFWLTHFVPLLLALCPSEPAHALAPGSSRVELFKRQNSTVPFEENGEVRQRVVHSFRLPALVNVDGVMVAIADARYDTSNDNSLIDTVVKYSVDDGETWETQIAIKNSRASSVSRVVDPTVIVKGNKIYVLVGSYNSSTSYWTSHGDARDWDILLAVGEVTKSIVDGKTTANITWGSPVSLKEFFPAYMEGMHTNQFLGGAGVAIVASNGNLVYPVQVTNKRKQVFSKIFYSEDDGKTWKFGKGRSDFGCSEPVALEWEGKLIINTRVDYRRRLVYESSDTEKPWVEAVGTLSRCWGPSPKSDQPGSQSSFTAVTIEGMRVMLFTHPLNFKGWWLRDRLNLWLTDNQRIYNVGQLSIGDENSAYSSVLYKDDKLYCLHEINSNEVYSLVFARLVGELRIIKSVLQSWKKWDSHLSSICTPADPAASSSERVCGPAVTTVGLAGFLSHSANKTKWKDAYRCVDASTANAERVSNGLKFAGVGGGALWPVSQQGQNQRYRFANHAFTLVASVTIHEAPSVASPLLGASLDSSGGKKLLGLSYDEKHQWQPIYGSTPVTPTGSWETGKRYHVVLTMANRNGSVYVDGELLKGSGQTVVPDRTPDISHFYVGGYGRSDMPTISHVTVNNVLLYNRQLNAEEIKTLFLSQDLIGTEAHMDSSSDSSAHGTPSTPVDSTAHGTPSTPADSSAHSTPSTPADSSAHSTPSTPVDSSAHSTPSTPADSSAHSTPSTPADSSAHSTPSTPVDSTAHGTPSTPADSSAHSTPSTPVDSSAHSTPSTPADSSAHSTPSTPVDSSAHSTPSTPADSSAHGTPSTPVDSSAHSTPSTPADSSANGTVLILPDGAALSTFSGGGLLLCACALLLHVFFTAVFF

SEQ ID NO: 3 - protein, 62 aa, Trypanosoma cruzi
MTTCRLLCALLALALCCCLXACTTANGGSTXSTPPSGTENKPAXGEAPSQPGASSGEAEASS

SEQ ID NO: 4 - protein, 68 aa, Trypanosoma cruzi
MEQERRQLLEKDPRRNAKEIAALEESMNARAQELAREKKLADRAFLDQKPERVPLADVPLDDDSDFVA

SEQ ID NO: 5 - protein, 227 aa, Trypanosoma cruzi
PPPFGQAAAGDKPSPFGQAAAGDKPPPFGQAAAGDKPSPFGQAAAGDKPPPFGQAAAGDKPSPFGQAAAGGKPPPFGQAAAGDKPSPFGQAAAGDKPPPFGQAAAGDKPSPFGQAAAGDKPPPFGQAAAGDKPPPFGQAAEGDKPPPFGQAAAGDKPAPFGQAAEGDKPPPFGQAAAADKPSLFGQAAAGDKLSLFGQAAAGDKPPPFGQAAEGDKPPPFGQAAAGD

SEQ ID NO: 6 - protein, 234 aa, Dirofilaria immitis
MKILFCFVLLAIAALRASVINRHNKRFAGFSVAGIGGTAGCVVVDNKLFANSFYLRDLTTEEQRELAQYVEDSNQYKEEVKTSLEERRKGWQLARHGEKDAKVLSSLAEKKFPKPPKKPSFCSAGDTTQYYFDGCMVQNNKIYVGRMYVRDLTSDEINQLKTFDAKMTAYQKYLSSSIQQQVDSLFGDKSNLFNLFTDTRHETSSQPSDATTISTTTQAPVEPPETPHFCIAIY

(19) United States
(12) Patent Application Publication
(10) Pub. No.: US 2025/0020642 A1
(43) Pub. Date: Jan. 16, 2025

(54) METHOD AND ANTIGEN COMPOSITION FOR RAPID DETECTION OF CHAGAS DISEASE, LEISHMANIASIS, AND/OR HEARTWORM IN HUMANS AND ANIMALS

(71) Applicant: VIDA PHARMACAL INC., Spring Branch, TX (US)

(72) Inventor: Alberto PANIZ-MONDOLFI, Spring Branch, TX (US)

(21) Appl. No.: 18/705,227

(22) PCT Filed: Oct. 28, 2022
(86) PCT No.: PCT/US2022/078838

§ 371 Date: Apr. 26, 2024

Related U.S. Application Data
(60) Provisional application No. 63/272,995, filed on Oct. 28, 2021.
(60) Provisional application No. 63/405,415, filed on Sep. 10, 2022.

(51) Int. Cl. G01N 33/543; G01N 33/58

(52) U.S. Cl. CPC G01N 33/54388; G01N 33/585; G01N 2333/4353; G01N 2333/44; G01N 2800/26

(57) ABSTRACT

Among several illnesses common to pets such as dogs that can also affect humans, American trypanosomiasis, commonly known as Chagas disease, is a parasitic 20 condition caused by the hemoflagellated protozoa, Trypanosoma cruzi. Active transmission is through triatomine vectors known as kissing bugs, which are prevalent in North and South America. Diagnostic methods, systems, test strips, test kits, and other immunoassay materials are provided for the detection of antibodies to one or more specific parasites of veterinary and human concern in biological samples, including those that are responsible for Chagas disease (Trypanosoma cruzi), Leishmaniasis (Leishmania infantum), and Heartworm (Dirofilaria immitis).

### CROSS REFERENCE TO RELATED APPLICATIONS

This application claims priority to U.S. provisional patent application no. 83/272,995, filed Oct. 28, 2021, and to U.S. provisional patent application no. 83/405,415, filed Sep. 10, 2022, the entire disclosures of each of which are incorporated herein by reference.

### FIELD

The present disclosure relates generally to the field of veterinary diagnostics, and more specifically to compositions and methods for determining antibodies against human and animal infectious parasitic diseases such as Chagas disease, Leishmaniasis, and/or Heartworm. Combinations of proteins from one more of Trypanosoma cruzi, Dirofilaria immitis, or Leishmania species are provided in immunoassay formats.

### BACKGROUND

Among several illnesses common to pets such as dogs that can also affect humans, American trypanosomiasis, commonly known as Chagas disease, is a parasitic condition caused by the hemoflagellated protozoa, Trypanosoma cruzi. Active transmission is through triatomine vectors known as kissing bugs, which are prevalent in North and South America. Defecation by infected insects during or after a blood meal passes infective trypomastigotes into the bite wound. Other routes of infection include transplacental transmission to offspring of infected pregnant humans and animals, consumption of a variety of infected foods such as juices and meats that have been tainted by the parasite and are contaminated with triatomine feces. Another source of these illnesses are blood transfusion from other patients that have not been screened for such parasites. After trypomastigotes enter the circulation, there is a brief period of replication and hematogenous spread through mononuclear cells, with the final destination being various tissues in the host, including the heart, fat, and the lymphatic system, where they reside and multiply as intracellular amastigotes. A century after its discovery, T. cruzi infection remains a serious health problem in humans and animals. It is a leading cause of cardiomyopathy that affects over 8 million people worldwide, and it is predicted that Chagas disease will cause at least 200,000 human deaths globally over the next 5 years, which is identical to the number of women living in the United States predicted to die of breast cancer in the same time period.

Whereas the highest prevalence of cardiomyopathy due to Chagas disease in people is found in Central and South American countries, studies show a widespread distribution of American trypanosomiasis in dogs in Texas, Louisiana, and Georgia and indicate that these southern states are the sites of important reservoirs for transmission of Trypanosoma cruzi in the United States. The prevalence of T. cruzi infection in stray and shelter-housed dogs has been reported to be as high as 7.5% to 8.8% and may exceed 50% in some working dog kennels in Texas. The pattern of disease progression in dogs with American trypanosomiasis is similar to that in people, which comprises acute, chronic asymptomatic, and chronic symptomatic phases. Acute disease occurs within 3 weeks after inoculation and centers around myocarditis with severe inflammation following the death of cardiac cells. Most affected dogs are <1 year of age, and clinical signs commonly include tachyarrhythmias, respiratory distress, lymphadenopathy, anorexia, diarrhea, neurologic abnormalities, peripheral edema, collapse, and sudden death. Parasitemia is profound and the mortality rate is high, especially among <1-year-old dogs.

The detection and treatment of Chagas diseases remains limited due to barriers and shortcomings of current diagnostic tests. In particular, Chagas disease diagnosis in veterinary medicine is currently limited commercially to antibody detection using an immunofluorescence assay (IFA). This approach uses whole epimastigote antigen from the disease vector and lends itself to false negatives due to variation in antigen across multiple strain types. There is no gold standard to test for T. cruzi infections in dogs, and the IFA is the only test approved for veterinary Chagas diagnostics. As recommended by the WHO for human Chagas diagnostics, reactivity on multiple serologic assays would be preferred to consider a dog T. cruzi-positive. Thus, Chagas infection in dogs is often overlooked in veterinary medicine, and despite a large prevalence in the U.S., there is currently no definitive testing protocol that can be carried out in a rapid manner so that the testing can be completed and the diagnosis made rapidly, such as during a regular doctor or veterinary appointment that may take upwards of 15 minutes to an hour.

Another disease that arises particularly in dogs is Dirofilariasis, commonly referred to as Heartworm, or Heartworm disease, which is a condition caused by the vector borne nematode Dirofilaria immitis. This disease is readily spread by mosquitoes which can introduce third-stage filarial larvae of Dirofilaria immitis into the skin of the definitive host, where they penetrate into the bite wound. In the definitive host, the L3 larvae undergo two more molts into L4 and adults. Adults reside in pulmonary arteries, and are occasionally found in the right ventricle of the heart. Adult females are usually 230-310 mm long by 350 μm wide; males are usually 120-190 mm long by 300 μm wide. Adults can live for 5-10 years. In the heart, the female worms produce microfilariae which circulate in peripheral blood. A mosquito ingests these microfilariae during a blood meal. After ingestion, the microfilariae migrate from the mosquito's midgut through the hemocoel to the Malpighian tubules in the abdomen. There the microfilariae develop into first-stage larvae and subsequently into third-stage infective larvae. The third-stage infective larvae migrate to the mosquito's proboscis and can infect another definitive host when the mosquito takes a blood meal. In humans, D. immitis larvae tend to follow the same migratory pathway as in the canine host, ending up in the lungs, where they often lodge in small-caliber vessels, causing infarcts and typical “coin lesions” visible on radiographs. Occasionally, D. immitis may cause subcutaneous or other ectopic infections.

The most typical hosts for D. immiffs are domestic dogs, coyotes, jackals, and wolves. Adult worms are occasionally found in others species such as domestic cats, bobcats, ferrets, and foxes. However, microfilaremia is usually low or absent in these aberrant hosts and thus they are not major reservoirs. Vectors include mosquitoes from several genera (Aedes, Culex, Anopheles, Mansonia). As a result, the American Heartworm Society recommends annual screening for all dogs over 7 months of age with both an antigen and a microfilaria test.

While the current generation of heartworm antigen tests identifies most “occult” (adult worms present but no circulating microfilariae) infections consisting of at least one mature female worm and are nearly 100% specific. Differences in sensitivity exist especially in cases with low worm burdens and/or low antigenemia. Currently there are no verified tests capable of detecting infections consisting of only adult male worms. As a result, there are substantial limitations of current heartworm tests in dogs. Among the most important is the notion of “low heartworm burden”. These would include numbers of female worms of 1-10, with the average heartworm infected dog having 15. Sensitivities plummet as low as 48% antigen detection when burdens are below 10. Also, due to the inherent properties of the antigen used to detect infection, a period of 7 months is required before diagnosis, setting the patient up for cardiovascular damage due to delay in diagnosis. False positives are also known to occur a) in well-type tests due to inadequate washing, b) due to residual circulating antigen post-adulticide treatment, and c) as a cross reaction with Spirocerca lupi or other unknown antigens. While the esophageal nematode S. lupi is most commonly encountered in the Southern states, such as Louisiana, this author has recently consulted on cases in Kansas, Nebraska and Missouri, one of which was positive on an antigen test.

Finally, a further condition arising in animals such as dogs and other mammals is Leishmania or Leishmaniasis. This is an illness affecting dogs, cats, horses, and humans, and has several forms including Cutaneous Leishmaniasis, most commonly due to L. braziliensis, and L. mexicana, as well as Visceral Leishmaniasis due to L. infantum infection. Once again, current methods to identify Leishmania infections have drawbacks, including the timing and the accuracy of the testing.

As a result, there has been a lack of adequate testing and detection of the above harmful parasitic diseases, and this is particularly a problem in those regions where humans and animals remain susceptible to infections and related illnesses caused by these parasites. There is thus ongoing need for improved approaches to detecting Chagas, Leishmaniasis, and Heartworm. The present disclosure in pertinent to this need.

### SUMMARY OF THE INVENTION

The present disclosure relates to overcoming disadvantages in the prior art, and to accommodate a need in the field by reducing false positive results and false negative results in diagnostic assays for diseases including Chagas, Leishmaniasis, and Heartworm, and in providing a rapid analysis for one, two, or all three of these conditions using sets of specific antigens.

Surprisingly it was found that this object can be met, and consequently disadvantages of the prior art can be overcome, by an improved diagnostic test for Chagas, Leishmaniasis, and Heartworm, wherein specific antigens and configurations of antigens and antibodies, and specific combinations of detection antibodies, are chosen for diagnostic tests which can detect one or more of these conditions. Using the present compositions of specific antigens in accordance with the present invention overcomes the disadvantages of previous approaches using single target antigens by providing testing redundancy. In addition, the described novel compositions (based on mixtures of antigens) targeting multiple pathogens of interests is comparable or superior in terms of specificity, sensitivity and reproducibility for detection of T. cruzi, Leishmania sp. and Dirofilaria immitis.

As discussed above, the present disclosure involves the use of specific antigens or combinations of antigens suitable for accurately detecting either specific or multiple parasites of veterinary and human concern in biological samples, including those that are responsible for Chagas disease (Trypanosoma cruzi), Leishmaniasis (Leishmania infantum and other Leishmania parasite species) and Heartworm (Dirofilaria immitis).

In particular, the present invention relates, in certain embodiments, to a rapid detection immunoassay method for qualitatively detecting the presence or absence of antibodies against multiple targets for one or more of a group of parasitic parasites including Trypanosoma cruzi, Leishmania species, and Dirofilaria immitis. The detection may be made using different biological matrices such as bodily fluid from a human or animal subject. In an embodiment, an antigenic composition comprising antigens is provided from a plurality of said target parasites; subsets containing antigens against a specific parasite so that individual subsets reflect a specific and different target parasite. Each of the subsets is then transferred to a test strip having parallel lanes so that individual lanes will have only antigens to a specific parasite, and a sample of a bodily fluid or other biological matrix obtained from a human or animal is applied to the test strip so that the presence or absence of antibodies against the target parasite can be determined using the described immunoassay methods.

The present disclosure also relates to a system for rapid detection in humans or animals of the presence or absence of parasite antibodies reflective of an infection from diseases caused by parasites of Trypanosoma cruzi, Leishmania species and Dirofilaria immitis via testing of a bodily fluid or other biological matrix from the human or animal subject. The immunoassay system comprises a test strip having parallel lanes coated with an antigenic composition comprising antigens from said target parasites wherein said antigenic composition is provided in separate antigenic composition subsets wherein each subset contains only antigens from a specific parasitic target, wherein each subset contains antigens from a different parasitic target, and wherein each subset is coated onto one of said parallel lane on said test strip so that a plurality of tests can be carried out at one time; a sample of a bodily fluid or other biological matrix from a human or animal subject that can be applied to said test strip; immunobinding detection reagents in each lane of said test strip which provide a signal that indicates immunobinding of the antigens when the sample contains antibodies against the target parasite(s); and a detection zone on the test strip wherein the signal indicative of immunobinding between the antigens on the test strip and antibodies against said target parasites can be observed. Any suitable signal that one skilled in the art would recognize as usable to detect the presence or absence of binding between an antigen and an antibody recognizing said antigen in an immunoassay may be used. For example, the immunoassay systems, methods, tests strips, and kits may utilize a detection signal selected from the group consisting of a colorimetric signal, a fluorescent signal, a chemiluminescent signal, a radioactive signal, and a visual signal.

The present disclosure also relates to test strips containing the antigenic compositions of the invention. In an embodiment, the test strips are configured so that separate parallel lanes contain an antigen subset that is exclusive to a particular parasite of interest which allows for the testing of multiple parasites at the same time. Test kits are also provided which include test strips and diagnostic reagents that can identify the presence or absence of parasite antibodies in the fluid sample of a human or animal subject by identifying the presence or absence of immunobinding between antigens on the test strip and the antibodies recognizing those antigens that reflect exposure to the particular parasite disease in the subject.

As indicated above, the invention is designed to particularly be useful for detection of the conditions of Chagas disease caused by the hemoflagellate parasite Trypanosoma cruzi; Leishmaniasis caused by different Leishmania species; and/or Heartworm disease caused by the nematode parasite Dirofilaria immitis. In exemplary embodiments, specific antigens for each condition are used in different antigenic combinations that improve the accuracy, sensitivity, efficiency, or a combination thereof, of the test results.

In an example, the disclosure provides a combination of materials, such as for use in a kit or device, comprising: a) a first substrate comprising at least one Trypanosoma cruzi antigen attached to the first substrate; and b) detectably labeled antibodies that are capable of specifically binding to antibodies produced by an animal that has been exposed to Trypanosoma cruzi. In certain examples, the at least one Trypanosoma cruzi antigen comprises at least one of Trypanosoma cruzi antigens that is an LA204 protein selected from the proteins in Table A, an IBMP-8 protein selected from the proteins of Table B, 1F8, Tc24, SAPA, B13, PEP2, FRA, TSSA, TcD, TcF, or TcE, or an antigenic fragment of any of said proteins, or a combination of said proteins, or a combination of said antigenic fragments, or a combination of said proteins and antigenic fragments thereof. In one example, the at least one Trypanosoma cruzi antigen comprises a combination of LA204 proteins, or antigenic fragments thereof. In one example, the at least one Trypanosoma cruzi antigen comprises all of the proteins of LA204 as in Table A. In one example, the at least one Trypanosoma cruzi antigen comprises the 1F8 protein, an IBMP-8 protein from Table B, or an antigenic fragment thereof. In one example, the at least one Trypanosoma cruzi comprises the 1F8 or an IBMP-8 protein.

In one example, for a test designed to separately test for antibodies from other described parasites, the described Trypanosoma cruzi material combinations above may be combined with: i) at least one Leishmania protein or antigenic fragment thereof attached to the first substrate or to a second substrate, and detectably labeled antibodies that are capable of specifically binding to antibodies produced by an animal that has been exposed to the Leishmania wherein the Leishmania, wherein said Leishmania protein is optionally rK39, rK28, rKR95, rK18, K39, K28, KR95, and K18; or ii) at least one Dirofilaria immitis antigen attached to the first substrate, the second substrate, or a third substrate and detectably labeled antibodies that are capable of specifically binding to antibodies produced by an animal that has been exposed to the Dirofilaria immitis; wherein said Leishmania antigen is optionally DIT33, ChimDiT33, PDi33, or antigenic fragments thereof. Combinations of i) and ii) are also included.

In any of the materials and methods described above, the substrate may be configured to permit flow of a liquid biological sample through the substrate. A representative and non-limiting example of such as substrate is nitrocellulose.

In any of the materials and methods described above, the detectably labeled antibodies comprise a detectable label that is visually detectable, and may be visually detectable by a human operator of the described combinations of materials. A non-limiting example of a detectable label is gold particles.

In any of the materials and methods described above, the detectably labeled antibodies may comprise polyclonal goat anti-feline antibodies. In non-limiting examples, the polyclonal goat anti-feline antibodies comprise anti-IgG and anti-IgM antibodies. In certain examples, the polyclonal goat anti-feline IgG and anti-IgM antibodies are capable of specifically binding to anti-feline and anti-canine antibodies that may be present in a biological sample. For use in detecting Trypanosoma cruzi antigens with the polyclonal goat anti-feline antibodies, the at least one Trypanosoma cruzi antigen comprises a combination of 1F8, Tc24, SAPA, B13, PEP2, FRA, TSSA, TcD, TcF, TcE, an LA204 protein selected from the proteins in Table B, and an IBMP-8 protein selected from the proteins of Table B. In one example, the at least one Trypanosoma cruzi antigen comprises all of the proteins as in Table A. In one example, the at least one Trypanosoma cruzi antigen comprises also comprises the 1F8 or the IBMP-8 or an antigenic fragment thereof. The described combinations of materials may further be in contact with biological sample obtained from a feline or canine, and thus the described assays while in operation are encompassed by the disclosure.

The described combinations of materials can be provided in the form of a kit. The kit includes comprises one or more sealed containers that contain the substrate and the one or more proteins or antigenic fragments thereof. The kit may also include one or more containers that contain the detectably labeled antibodies, and may include printed material that identifies the combination of materials and/or provides instructions to a user for using the combination of materials to determine antibodies from a subject.

The disclosure also provides a method comprising exposing a biological sample obtained from a mammal to any of the described combination of the materials, and determining a signal from the detectably labeled antibodies. In embodiments, the biological sample is obtained from a canine or feline animal. In embodiments, a signal from the detectably labeled antibodies signifies the animal has been exposed to Trypanosoma cruzi, which may aid in a determination that the that the animal has Chagas disease. The disclosure further optionally includes administering to the animal at least one therapeutic agent for treatment of the Chagas disease.

The disclosure also includes individual and combined tests for each of Leishmania and heartworm. The method comprises exposing a biological sample obtained from an animal to a material that has Leishmania antigens, Dirofilaria immitis antigens, or a combination thereof, and determining a signal from the detectably labeled antibodies. In embodiments, this approach is used with canine or feline animals. In embodiments, this approach indicates that the animal has been exposed to the particular parasite from which the biological sample was obtained. In embodiments, determining the detectably labeled antibodies aids in a determination that the mammal has Leishmaniasis, or aids in a determination that the animal has heartworm. The disclosure optionally includes administering therapeutic agents to animals that have been determined to have Leishmaniasis or heartworm. For one individual parasite test, the disclosure includes a combination of materials comprising: a) a substrate comprising at least one Leishmania antigen attached to the substrate, wherein said antigen is optionally selected from Leishmania protein rK39, rK28, rKR95, rK18, K39, K28, KR95, and K18, and antigenic fragments thereof; and b) detectably labeled antibodies that are capable of specifically binding to antibodies produced by an animal that has been exposed to Leishmania. For another individual test, the disclosure provides combination of materials comprising: a) a substrate comprising at least one at least Dirofilaria immitis antigen attached to the substrate, wherein said antigen is optionally selected from DIT33, ChimDiT33, PDi33, and antigenic fragments thereof; and b) detectably labeled antibodies that are capable of specifically binding to antibodies produced by an animal that has been exposed to the Dirofilaria immitis.

### BRIEF DESCRIPTION OF THE DRAWINGS

FIG. 1. Three representative configurations of a lateral flow assay.

FIG. 2. Representative Chagas detection reagents.

FIG. 3, panel A. Representative negative Chagas lateral flow result. Panel B, representative positive Chagas lateral flow result Positive result is boxed.

FIG. 4. Representative heartworm detection reagents.

FIG. 5. Representative Leishmania detection agents.

### DETAILED DESCRIPTION

Unless defined otherwise herein, all technical and scientific terms used herein have the same meaning as commonly understood by one of ordinary skill in the art to which this disclosure pertains.

Unless specified to the contrary, it is intended that every maximum numerical limitation given throughout this description includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification includes every narrower numerical range that falls within such broader numerical range, as if such narrower numerical ranges were all expressly written herein.

The singular forms “a”, “an” and “the” include plural referents unless the context clearly dictates otherwise. The terms “a” (or “an”), as well as the terms “one or more,” and “at least one” can be used interchangeably herein. Furthermore, “and/or” where used herein is to be taken as specific disclosure of each of the two specified features or components with or without the other.

All amino acid sequences of the proteins and peptides described herein include sequences having 90.0-99.9% identity across their entire lengths to the described amino acid sequences. The amino acid sequence associated with each database entry of this disclosure is incorporated herein by reference as presented in the database on the effective filing date of this application or patent. The database entries include but are not necessarily limited to the UniProt and GenBank databases.

Aspects of this disclosure include each protein described herein, and all combinations of such proteins. All amino acid sequences in this disclosure include all segments thereof that are at least 5 contiguous amino acids of the described sequences. The disclosure thus includes antigenic fragments of the described protein. An antigenic fragment means a segment of a described protein that stimulates an antibody response in a host that is exposed to a protein that comprises the segment. The proteins and/or antigenic fragments of the proteins are used in assays as further described herein to provide profiles of antibodies that can bind with specificity to the described proteins or antigenic fragments thereof. In certain instances the proteins and antigenic fragments described herein may themselves be referred to as antigens.

Determination of the antibodies that bind to the described proteins or antigenic fragments thereof (wherein said binding is not necessarily mutually exclusive) can be performed using any suitable approach, including but not limited to any type of enzyme-linked immunosorbent assay (ELISA) assay, including but not limited to a direct ELISA, a sandwich ELISA, a competitive ELISA, and a reverse ELISA. In embodiments, one or proteins and/or peptides described herein can be incorporated into an immunodiagnostic device, such as a microfluidic device, a lateral flow device, and the like. Non-limiting combinations of materials for use in such assays are described further below.

In non-limiting embodiments, the disclosure provides for a device configured to generate a signal that is based on antibodies bound to one or more of the described proteins and/or antigenic fragments thereof. In embodiments, the signal is optically accessible. In embodiments, at least some antibodies, if present in the biological sample, are bound to at least some of the proteins or antigenic fragments thereof in the plurality of proteins. In embodiments, the antibodies are bound to the proteins or antigenic fragments thereof or a combination thereof, and the assay further comprises detectably labeled antibodies bound to the antibodies that are bound to the proteins or antigenic fragments thereof, or a combination thereof. Thus, in one approach, the disclosure includes determining a signal from the detectably labeled antibodies. The detection antibodies or other detection agent can be labeled using any suitably detectable label. In embodiments, the detectable labels produce a signal that comprises UV light (<380 nm), visible light (380-740 nm) or far red light (>740 nm). In embodiments, the detectable signal comprises a fluorescent signal. In embodiments, relative light units (RLUs) are used. In embodiments, a cell-based assay can be used in the methods of the disclosure. In embodiments, a suitable laser may be used. In embodiments, detection, e.g., secondary antibodies, may be conjugated to a gold particle, such as a gold nanoshell. In embodiments, a gold nanoparticle is used, and may be adsorbed to the detection antibodies, and therefore in certain embodiments the gold nanoparticle is not covalently attached to the detection antibodies. Suitable gold nanoparticles for use in the described approaches are commercially available.

In embodiments, and while certain implementations of the described approach may be interpretable by a human user of a described device, such as a lateral flow device, in other embodiments, an imager may be used and located proximal to analyzed sample. In embodiments, free-space optics may be used to detect a signal from the assay using any suitable signal detection device that is placed in proximity to the location where a detectable signal is generated, such as a CCD camera. In embodiments, the disclosure provides a microfluidic device for use in sample analysis. In embodiments, the microfluidic device may comprise, among other features, an optical waveguide to transmit a signal to any suitable measuring device such that optical accessibility to sample is not necessarily required to detect the signal. In embodiments, lens-less optics, and/or a cell phone based imaging approach is used. In embodiments, one or more segments of an assay device on which the described antibody analysis is performed can be connected to or in communication with a digital processor and/or a computer running software to interpret the presence, absence, amount, or a ratio of antibodies that bind to one or more of the described peptides. The processor may run software and/or implement an algorithm to interpret an optically detectable signal, and generate a machine and/or user readable output. In an embodiment, an assay device used to perform the antibody analysis can be integrated or otherwise inserted into an adapter that comprises a detection device, such as a camera, or a microscope, including but not limited to a fluorescent microscope. In embodiments, a computer readable storage medium can be a component of an assay device of this disclosure, and can be used during or subsequent to performing any assay or one or more steps of any assay described herein. In embodiments the computer storage medium is a non-transitory medium, and thus can exclude signals, carrier waves, and other transitory signals.

In embodiments, the one or more of the described proteins and/or antigenic fragments thereof are used in a device referred to in the art as a “BioCD” device, and thus detectable labels may not be required. In embodiments, the BioCD is a differential phase-contrast BioCD biosensor, such as that described in “Differential phase-contrast BioCD biosensor,” Appl Opt. (2007) 46:8196-6209, from which the entire description is incorporated by reference. In embodiments, the presently described antigens are used in a device and/or method as described in U.S. patent U.S. Pat. Nos. 10,100,092, 7,910,356, 7,787,126, and 7,683,092, from which the disclosures of devices for use in immunoassays, and methods of using the devices, are incorporated herein by reference.

In embodiments, an assay of this disclosure may include an antigen component that comprises or consists of antigens that are described herein. Thus, in embodiments, one or a combination of the described antigens may be the only antigens in the assay. In embodiments, the antigens used in a described antibody analysis may comprises or consist of at least two different antigens. In embodiments, the antigens used in a described antibody analysis may comprises or consist of 2-1,000 antigens, inclusive, and including all numbers and ranges of numbers there between.

In embodiments, the described proteins and antigenic fragments thereof can be modified using any suitable approach, including but not limited to including one or more purification tags, including but not limited to a His-tag. In an embodiment, a His-tag is a linear sequence of n histidine residues where n is typically 6-10. His-tags achieve purification by binding specifically to nickel or cobalt ions, which may be for example, attached to a substrate, such as any suitable beads. The His-tag, or any other suitable purification tag, may be placed at the N-terminus, or at the C-terminus of the protein or peptide. In embodiments, a FLAG-tag, or FLAG octapeptide, or FLAG epitope, may be included. Suitable FLAG sequences are known in the art. In embodiments, a Small ubiquitin-related modifier (SUMO) tag, such as a His-SUMO tag can be included.

In embodiments, one or more of the proteins or antigenic fragments thereof may comprise a linker. The term “linker” refers to a chemical moiety that connects a segment the peptide to another material. Linkers include amino acids, but other linkers are encompassed as well. Generally speaking, amino acid linkers may be principally composed of relatively small, neutral amino acids, such as Glycine, Serine, and Alanine, and can include multiple copies of a sequence enriched in Glycine and Serine. In embodiments, the linker is provided as a means to connect the protein or peptide to a substrate. As such, the linker may be attached to a binding partner that connects the peptide to a substrate.

In embodiments, one or more of the antigenic fragments thereof are modified so that they can be attached to a substrate. The attachment may or may not be via a linker. In various embodiments, the one or more antigenic fragments thereof can be reversibly or irreversibly attached to a substrate, such as by being covalently, ionically, or physically bound to a, for example, a solid-phase substrate using methods such as covalent bonding via an amide or ester linkage, ionic attraction, or by adsorption, non-limiting examples of which comprise biotin and streptavidin tags. The substrate can be any suitable substrate onto which one or more described proteins or antigenic fragments thereof can be attached. Examples include substrates typically used in immunodetection assays, lateral flow devices, bead-based assays, nanoparticles such as gold nanoparticles, microfluidic devices, etc. Thus, a solid substrate can be a porous solid substrate that allows the flow of liquid through the substrate, such as by capillary action. In a non-limiting embodiment, the substrate comprises nitrocellulose. In embodiments, the substrate may be a film. In embodiments, the substrate may comprise one or more channels. The substrate may be in fluid communication with one or more wicking materials, such as a woven mesh or cellulose fiber, such as for use as a sample application pad, and may further be in fluid communication with a suitable filter material to separate non-antibody material from the biological sample.

The biological liquid sample can flow through the porous substrate via any suitable means, such as by capillary action, microfluidics, etc. The substrate can also be a non-porous solid substrate, such as beads formed from glass or other non-porous materials, such as plastic. In certain embodiments, the substrate, the proteins or antigenic fragments thereof may be functionalized to facilitate their attachment. The substrate may be blocked prior to attaching the proteins and/or antigenic fragments thereof to reduce artifacts. The describes assays may also be performed in solution.

The disclosure includes lateral flow devices that include one or more substrate strips, and thus may be provided in the form of a cassette. A single strip, or separate strips, may be provided and may comprise proteins or antigenic fragments thereof to facilitate detection of antibodies that bind to Chagas antigens, heartworrn antigens, Leishmaniasis antigens, and combinations thereof. The single strip, or a separate strip, may also comprise a control to provide a signal that the particular assay is functional. The described assays may be qualitative or quantitative. Thus, the described assays can provide a determination of the presence of antibodies, the absence of antibodies, or an amount of antibodies that are present in a particular biological sample. In certain embodiments a multiplex assay is provided, wherein antigens from more than one of the described infectious agents can be combined in a single assay, in which case the disclosure provides for use of separately detectable labels.

In embodiments, one or more of the antigenic fragments thereof are present in vitro, such as in an in vitro assay. In embodiments, the proteins or antigenic fragments thereof and are in contact with a biological sample obtained from an individual, such as any mammal, representative embodiments of which include but are not limited to human, canines, felines, and equine animals. In embodiments, the biological sample is from an animal who has been exposed or is suspected of having been exposed, or was at risk of exposure, to one or causative agents of Chagas disease, Heartworm, or Leishmaniosis.

In certain embodiments a result based on a determination of the presence, absence, amount, type, ratio, or a combination thereof for antibodies analyzed using an approach and/or a device of this disclosure is obtained and is fixed in a tangible medium of expression, such as a digital file, and/or is saved on a portable memory device, or on a hard drive, or is communicated to a web-based or cloud-based storage system. The determination can be communicated to, for example, a veterinary health care provider for the purpose of making a diagnosis, and/or recommending or not recommending any particular medical intervention. In certain non-limiting embodiments, based on a determination of the presence of antibodies, the disclosure further comprises administering to the subject from which the biological sample was obtained a suitable therapeutic agent. For example, for Leishmaniasis, the disclosure includes administering any of Liposomal amphotericin B, pentamidine, Ivermectin, antimony, amphotericin, paromomycin and miltefosine. In embodiments, for Chagas, the disclosure includes administering any of benznidazole, nifurtimox, and itraconazole. In embodiments, for Heartworm, the disclosure includes administering melarsomine.

In certain examples the disclosure comprises an article of manufacture, which in embodiments can also be considered kits. The kits can comprise a combination of materials as described herein. The article of manufacture comprises at least one component for use in the antibody analysis described herein, and packaging. The packaging can contain any proteins or antigenic fragments thereof or combinations thereof described herein, and may further provide reagents for use in determining antibodies, and/or for sample collection. The kit can be provided as a component of a cartridge or similar component, or for example a multi-well plate. Such components may be provided pre-loaded with any combination of reagents required to perform the described analysis. Such components may be provided with the substrate and certain reagents as a lyophilized form for reconstitution in, for example, a suitable immunodiagnostic buffer. In various embodiments, the article of manufacture includes printed material. The printed material can be part of the packaging, or it can be provided on a label, or as paper insert or other written material included with the packaging. The printed material provides information on the contents of the package, and instructs a user how to use the package contents for antibody analysis, and may provide information about determination of a property functioning assay and a determination of the presence, absence, and or amount of antibodies present in a sample.

In certain embodiments a result obtained from using a method and/or device and/or system and/or a combination of materials of this disclosure can be compared to a suitable reference, examples of which include but are not limited to control sample(s), a standardized curve(s), and/or experimentally designed controls such as a known input value. Any signal used for determining the described antibodies can be normalized to account for, for instance, differing protein lengths which may comprises a different number of epitopes relative to other proteins that may be used in the assay, thereby potentially producing more antibodies bound to a longer protein relative to a shorter protein.

In embodiments, a result produced using an approach of this disclosure is improved relative to a result obtained for an immunological assay for the same antibodies, but that is performed using different reagents than those used in the described assays of this disclosure. In an embodiment, an improved result comprises improved specificity, improved sensitivity, or a combination thereof, relative to a comparative assay. In embodiments, a comparative assay comprises an IFA assay. In embodiments, a comparative assay comprises an immunological assay that uses a capture antibody, instead of a configuration wherein an antigen is attached directly to a substrate. In an embodiment, the improved result is obtained using detectably label anti-feline IgG antibodies, detectably label anti-feline IgM antibodies, or a combination thereof, as detection antibodies for analysis of biological samples, including but not necessarily limited to canine samples. In embodiments, the detectably label anti-feline IgG and IgM are antibodies produced by a goat. In non-limiting embodiments, sensitivity is improved relative to a comparative assay by at least 50% to 75%. In an embodiment, improved sensitivity means a described assay produces fewer false negative results than a comparative assay. In an embodiment, improved specificity means a described assay produces fewer false positive results than a comparative assay. In embodiments, a result obtained by performing a method of the disclosure may be obtained faster than a result obtained by using a comparative assay.

Representative Chagas antigens that are suitable for use in the described assays include but are not necessarily limited to 1F8, GenBank accession no. CAA26599.1; Tc24 GenBank accession no ABA62611.1, SAPA, UniProt no. 000773, B13, GenBank accession no. AAP88022.1, PEP2, GenBank accession no. AAP88022.1, TcD, GenBank accession no. EAN98335.1, TcF, FRA, TcE, LA204, and IMBP-8 proteins. LA204 is a commercially available mixture of T. cruzi proteins, available for example, from East Coast Bio, under Catalog number LA204. The proteins are a mixture of proteins from T. cruzi proteins strains M/HOM/BR/78/SYLVIO-X10 CL, M/HOM/AR/74/CA-1 and Texas-Tulane. The present disclosure includes a characterization of the proteins in the LA204 product. The proteins are provided in Table A, which provides UniProt database entries for each protein in the LA204 mixture. GenBank accession numbers for IMBP-8 proteins are provided in Table B.

TABLE A
LA204 UniProt Accession Numbers

| | | | |
|---|---|---|---|
| A0A2V2X5H0 | A0A2V2XQW2 | A0A2V2VYQ3 | A0A2V2WFC2 |
| A0A2V2W077 | A0A2V2WVA1 | A0A2V2WP51 | A0A2V2WI81 |
| A0A2V2UXF5 | A0A2V2WXY6 | A0A2V2XE27 | A0A2V2WDW5 |
| A0A2V2VTM0 | A0A2V2V7B1 | A0A2V2XQF0 | A0A2V2WSY4 |
| A0A2V2VS10 | A0A2V2WL30 | A0A2V2XMM3 | A0A2V2WR94 |
| A0A2V2V8X8 | A0A2V2WRW1 | A0A2V2XMN2 | A0A2V2WD65 |
| A0A2V2VQY5 | A0A2V2WVR0 | A0A2V2W9C0 | A0A2V2WXE3 |
| A0A2V2V595 | O61107 | A0A2V2W620 | A0A2V2X903 |
| A0A2V2V065 | A0A2V2V2P8 | A0A2V2XHH0 | A0A2V2WKA0 |
| A0A2V2V5M4 | A0A2V2W0A9 | A0A2V2WMN6 | A0A2V2WM02 |
| A0A2V2UXG4 | O96763 | A0A2V2WGK0 | A0A2V2VZJ3 |
| A0A2V2V7F4 | A0A2V2W573 | A0A2V2WKR7 | A0A2V2UUI6 |
| A0A2V2V9I0 | A0A2V2W206 | A0A2V2WJH9 | A0A2V2VK35 |
| A0A2V2X840 | A0A2V2XGT6 | A0A2V2WVA0 | A0A2V2W514 |
| A0A2V2X6K9 | A0A2V2WWC6 | Q598P5 | A0A2V2XN58 |
| A0A2V2VSW7 | A0A2V2WVA9 | A0A2V2VYM4 | A0A2V2WU47 |
| A0A2V2XFA6 | A0A2V2WV86 | A0A2V2VX35 | A0A2V2WQC6 |
| A0A2V2WFD8 | A0A2V2VVI5 | A0A2V2WVE2 | A0A2V2XHX1 |
| A0A2V2WFC3 | A0A2V2VS74 | A0A2V2VAS6 | A0A2V2XAM0 |
| A0A2V2WKB2 | A0A2V2VTW4 | A0A2V2WGT5 | A0A2V2XD83 |
| A0A2V2UP60 | A0A2V2WXC8 | A0A2V2W2R9 | A0A2V2XAL8 |
| A0A2V2WGJ8 | A0A2V2XBW8 | A0A2V2WFU7 | A0A2V2XAK1 |
| A0A2V2WHD2 | A0A2V2VB61 | A0A2V2XEH3 | A0A2V2WXM7 |
| A0A2V2X955 | A0A2V2W4S9 | A0A2V2WBL0 | A0A2V2WX48 |
| A0A2V2UK30 | A0A2V2WXN9 | A0A2V2X3E4 | A0A2V2W8P4 |
| Q8STF3 | A0A2V2WLD2 | A0A2V2VC99 | A0A2V2W2W5 |
| Q9U664 | A0A2V2WNZ0 | O79469 | A0A2V2W8C8 |
| A0A2V2WC93 | A0A2V2XLG7 | A0A2V2WRH3 | A0A2V2WW41 |
| A0A2V2XES3 | A0A0F6VXC3 | A0A2V2X9R4 | A0A2V2WY93 |
| A0A2V2XFM8 | A0A0F6YE11 | A0A2V2X2Y0 | A0A2V2XAR8 |
| A0A2V2WXT0 | A0A0F6VXL3 | A0A2V2WSN9 | A0A2V2X9Y6 |
| A0A2V2W5G7 | A0A2V2WPQ7 | O61101 | A0A2V2VM67 |
| A0A2V2X924 | A0A2V2XFI7 | A0A2V2XBY3 | A0A2V2W7E1 |
| A0A2V2WJH7 | A0A2V2WVS0 | A0A2V2VMU4 | A0A2V2WKQ0 |
| A0A2V2XFZ1 | A0A2V2UY29 | A0A2V2X389 | A0A2V2WJU3 |
| A0A2V2X6X5 | A0A2V2W7J4 | A0A2V2V6P9 | A0A2V2XCK3 |
| A0A2V2WP61 | A0A2V2WFJ9 | A0A2V2X730 | A0A2V2XKR0 |
| A0A2V2XC14 | A0A2V2X547 | A0A2V2X897 | A0A2V2UX62 |
| A0A2V2XLY2 | A0A2V2WX06 | A0A2V2UIF3 | A0A2V2X4L6 |
| Q8T9X5 | A0A2V2X035 | A0A2V2WIX9 | A0A2V2WGS3 |
| A0A2V2WNP4 | A0A2V2W2P0 | A0A2V2X7U8 | A0A2V2WZQ3 |
| A0A2V2XDJ5 | A0A2V2W3G6 | A0A2V2WL05 | A0A2V2WGK6 |
| A0A2V2WHR1 | A0A2V2VVS5 | A0A2V2VMN6 | A0A2V2W7L0 |
| A0A2V2W436 | A0A2V2VBM7 | A0A2V2WDX6 | A0A2V2W0T8 |
| A0A2V2W6T3 | A0A2V2WNK9 | A0A2V2WQI7 | A0A2V2WWN1 |
| A0A2V2W9L5 | A0A2V2VQ67 | A0A2V2WA05 | A0A2V2VK45 |
| A0A2V2WAS9 | A0A2V2WIH3 | A0A2V2WEI3 | A0A2V2VMZ2 |
| A0A2V2WCN3 | A0A2V2XEW2 | A0A2V2WET0 | A0A2V2VKE2 |
| A0A2V2W7F9 | A0A2V2VKB4 | A0A2V2WEQ9 | A0A2V2WVL8 |
| A0A2V2V225 | A0A2V2VKL3 | A0A2V2W927 | A0A2V2W0Q8 |
| A0A2V2WEF6 | A0A2V2X690 | A0A2V2VI28 | A0A2V2WRS5 |
| A0A2V2WH08 | A0A2V2VMC7 | A0A2V2WDM4 | A0A2V2XM29 |
| A0A2V2VWT8 | A0A2V2X495 | A0A2V2XMC1 | A0A2V2W5V1 |
| A0A2V2WQE2 | A0A2V2W9M2 | A0A2V2WQX7 | A0A2V2W5U0 |
| A0A2V2W6D7 | A0A2V2WR11 | A0A1P8CYY9 | A0A2V2W875 |
| A0A2V2UU67 | A0A2V2WM88 | A0A2V2WST8 | A0A2V2X126 |
| A0A2V2WJ51 | A0A2V2UI35 | A0A2V2WRS1 | A0A2V2XEE3 |
| A0A2V2XJE7 | A0A2V2WCK6 | A0A2V2VYI2 | A0A2V2WRL2 |
| A0A2V2VXF5 | A0A2V2VZ45 | A0A2V2WQK9 | A0A2V2V2R6 |
| A0A2V2WEQ5 | A0A0C5BV55 | A0A2V2W9A4 | A0A2V2WJU5 |
| A0A2V2WM76 | A0A2V2VT40 | Q9U4H7 | A0A2V2XL43 |
| A0A2V2WYD4 | A0A2V2WFL7 | A0A2V2W9J5 | A0A2V2V8E2 |
| A0A2V2VUY0 | A0A2V2WKD4 | A0A2V2W3N3 | A0A2V2VSW1 |
| A0A2V2WMM5 | A0A2V2WHM8 | A0A2V2W6N7 | A0A2V2VX20 |
| A0A2V2W6U3 | A0A2V2XJU3 | A0A2V2WHI3 | A0A2V2UPG1 |
| A0A2V2W124 | A0A2V2XGR5 | A0A2V2X9L5 | A0A2V2X592 |
| A0A2V2VLR7 | A0A2V2WCK3 | A0A2V2XL39 | A0A2V2W0U8 |
| A0A2V2W9K3 | A0A2V2WI80 | A0A2V2VDJ1 | A0A2V2XAD7 |
| A0A2V2WI78 | A0A2V2VFG7 | A0A2V2WC16 | A0A2V2XNF2 |
| A0A2V2WYE4 | A0A2V2WL33 | A0A2V2WFX0 | A0A2V2XBA1 |
| A0A2V2WZD6 | A0A2V2V7C6 | A0A2V2WFS6 | A0A2V2XC82 |
| A0A2V2W647 | A0A2V2V107 | A0A2V2X0V0 | A0A2V2XMX4 |
| A0A2V2XMX9 | A0A2V2W4G1 | A0A2V2WY72 | A0A2V2WG23 |
| A0A2V2XQF5 | A0A2V2XWV4 | A0A2V2WRG9 | A0A2V2VED7 |
| A0A2V2WLH6 | A0A2V2WLA3 | A0A2V2X5F4 | A0A2V2X4H7 |
| A0A2V2XBN4 | A0A2V2X9F6 | A0A2V2XB80 | A0A2V2XAM3 |
| A0A2V2V2A6 | A0A2V2X8V0 | A0A2V2X693 | A0A2V2X4K8 |
| A0A2V2VXQ0 | A0A2V2X6V0 | A0A2V2WTB1 | A0A2V2WR87 |
| A0A2V2X7D2 | A0A2V2X6W9 | A0A2V2XAE9 | A0A2V2X4C2 |
| A0A2V2W5U5 | A0A2V2XCQ1 | A0A2V2VL08 | A0A2V2WL35 |
| A0A2V2XFF6 | A0A2V2W742 | A0A2V2WMF3 | A0A2V2UIM7 |
| A0A2V2WF83 | A0A2V2X287 | A0A2V2X931 | A0A2V2UZD1 |
| A0A2V2WUX2 | A0A2V2X3D7 | A0A2V2WEA8 | A0A2V2VXH5 |
| A0A2V2WVF8 | A0A2V2X2E6 | A0A2V2XC91 | A0A2V2X2D7 |
| A0A2V2V5E0 | A0A2V2WAA9 | A0A2V2V649 | A0A2V2X001 |
| A0A2V2WSZ7 | A0A2V2XFQ7 | A0A2V2V3D8 | A0A2V2WCT4 |
| A0A2V2WTW6 | A0A2V2W6G9 | A0A2V2W9F6 | A0A2V2USR4 |
| A0A2V2X5L9 | A0A2V2XM43 | A0A2V2VCM7 | A0A2V2XKK4 |
| A0A2V2WTU9 | A0A2V2W5I8 | A0A2V2VCN8 | A0A2V2VJ68 |
| A0A2V2WFS8 | A0A2V2WAM0 | A0A2V2XK99 | A0A2V2WM91 |
| A0A2V2X280 | A0A2V2XB96 | A0A2V2WV32 | A0A2V2VQ92 |
| A0A2V2UWQ7 | A0A2V2WC19 | A0A2V2W4G5 | A0A2V2V886 |
| A0A2V2XDH3 | A0A2V2X905 | A0A2V2WW92 | A0A2V2VDA5 |
| A0A2V2WDM9 | A0A2V2V3J8 | A0A2V2WUH5 | A0A2V2VZX2 |
| A0A2V2WIE0 | A0A2V2XHI9 | A0A2V2WS51 | A0A2V2UXA2 |
| A0A2V2WDF8 | A0A2V2XGM1 | A0A2V2WY83 | A0A2V2VU16 |
| A0A2V2W739 | A0A2V2WQS8 | A0A2V2WZA8 | A0A2V2V047 |
| A0A2V2WT05 | A0A2V2XHI4 | A0A2V2VYR4 | A0A2V2WRG6 |
| A0A2V2WD75 | A0A2V2XAT8 | A0A2V2XPS2 | A0A2V2VJB1 |
| A0A2V2X0K2 | A0A2V2X9V2 | A0A2V2XIA1 | A0A2V2W912 |
| A0A2V2WRR4 | A0A2V2WKH0 | A0A2V2VMT5 | A0A2V2WRB8 |
| A0A2V2WT00 | A0A2V2WKL4 | A0A2V2X2F9 | A0A2V2W3H4 |
| A0A2V2WSM6 | A0A2V2V954 | A0A2V2VZF9 | A0A2V2VWH8 |
| A0A2V2W7Q1 | A0A2V2WLG6 | A0A2V2VK70 | A0A2V2WFG4 |
| A0A2V2WGT9 | A0A2V2XAZ8 | A0A2V2W5W6 | A0A2V2V2J0 |
| A0A2V2WJF8 | A0A2V2XLX1 | A0A2V2VA43 | A0A2V2VWI8 |
| A0A2V2WGT8 | A0A2V2VTM7 | A0A2V2WX92 | A0A2V2X7B6 |
| A0A2V2WGE5 | A0A2V2WS20 | A0A2V2WXK5 | A0A2V2VXX0 |
| A0A2V2WLN5 | A0A2V2WBQ8 | A0A2V2V7W5 | A0A2V2WLG3 |
| A0A2V2WF10 | A0A2V2V0Z6 | A0A2V2XKU0 | A0A2V2WEF8 |
| A0A2V2VB53 | A0A2V2W9W8 | A0A2V2XKV0 | A0A2V2V2N6 |
| A0A2V2XCC4 | A0A2V2WKY3 | A0A2V2WH20 | A0A2V2X3N5 |
| A0A2V2X6H7 | A0A2V2V4F9 | A0A2V2WRI2 | A0A2V2V383 |
| A0A2V2UV35 | A0A2V2W0L1 | A0A2V2VV01 | A0A2V2VQ98 |
| A0A2V2X7F0 | A0A2V2W9V5 | A0A2V2WFS0 | A0A2V2X188 |
| A0A2V2W9E6 | A0A2V2VXD6 | A0A2V2WEF9 | A0A2V2WBP7 |
| A0A2V2X6G0 | A0A2V2WJC9 | A0A2V2W610 | A0A2V2WDB3 |
| A0A2V2W6S6 | A0A2V2WQF0 | A0A2V2VIP9 | A0A2V2XH35 |
| A0A2V2X6H3 | A0A2V2X390 | A0A2V2W5T3 | A0A2V2WCE5 |
| A0A2V2W707 | A0A2V2W0R5 | A0A2V2WWX1 | A0A2V2XHB0 |
| A0A2V2W8Z2 | A0A2V2W0N7 | A0A2V2V0Y7 | |
| A0A2V2X010 | A0A2V2W8L1 | A0A2V2X3N9 | |
| A0A2V2XHL0 | A0A2V2WXK6 | A0A2V2W9W7 | |
| A0A2V2WCJ2 | A0A2V2WTG4 | A0A2V2WFV0 | |
| A0A2V2WLH8 | A0A2V2XK70 | A0A2V2WIN9 | |
| A0A2V2VU90 | A0A2V2XL44 | A0A2V2VT05 | |
| A0A2V2WTS4 | A0A2V2WH83 | A0A2V2VYP6 | |
| A0A2V2WSE3 | A0A2V2VLR5 | A0A2V2UQP5 | |
| A0A2V2WGY6 | A0A2V2VJE5 | A0A2V2W307 | |
| A0A2V2VXG6 | A0A2V2VZ90 | A0A2V2WAI7 | |
| A0A2V2X9R8 | A0A2V2X8P9 | A0A2V2W9D3 | |
| A0A2V2V3Q1 | A0A2V2WL51 | A0A2V2UGY1 | |
| A0A2V2VQE9 | A0A2V2W5U4 | A0A2V2W4J4 | |
| A0A2V2UJI6 | A0A2V2WNT7 | A0A2V2V115 | |
| A0A2V2V9A1 | A0A2V2V6K2 | A0A2V2UXF1 | |
| A0A2V2WLH5 | A0A2V2UXF9 | A0A2V2VFZ8 | |
| A0A2V2XB07 | A0A2V2VXW5 | A0A2V2UWQ9 | |
| A0A2V2VY00 | A0A2V2WMH6 | A0A2V2W4M3 | |
| A0A2V2W959 | A0A2V2VTK0 | A0A2V2W5J1 | |
| A0A2V2VN74 | A0A2V2W905 | A0A2V2UU13 | |
| A0A2V2US66 | A0A2V2W363 | A0A2V2WZ84 | |
| A0A2V2WKK4 | A0A2V2VLV1 | A0A2V2XGP0 | |
| A0A2V2WEC6 | A0A2V2V9P3 | A0A2V2W7Y4 | |
| A0A2V2WZQ4 | A0A2V2V792 | A0A2V2WXP9 | |
| A0A2V2W439 | A0A2V2WIA5 | A0A2V2VTN9 | |
| A0A2V2WKS1 | A0A2V2VQ78 | A0A2V2VHB5 | |
| A0A2V2XK59 | A0A2V2WA02 | A0A2V2US31 | |
| A0A2V2XJZ6 | A0A2V2WHM0 | A0A2V2VEW9 | |
| A0A2V2XI18 | A0A2V2W751 | A0A2V2VMV2 | |
| A0A2V2VW96 | A0A2V2W6T1 | A0A2V2WH10 | |
| A0A2V2UVM3 | A0A2V2X3E1 | A0A2V2WY15 | |
| A0A2V2WHC7 | A0A2V2V5F8 | A0A2V2X7W1 | |
| A0A2V2UNK2 | A0A2V2V600 | A0A2V2VLU9 | |
| A0A2V2WEF5 | A0A2V2V6C4 | A0A2V2WRA8 | |
| A0A2V2V316 | A0A2V2X102 | A0A2V2WTQ5 | |
| A0A2V2XGA3 | A0A2V2VN84 | | |
| A0A2V2XFC3 | A0A2V2VN66 | | |
| A0A2V2XFE2 | A0A2V2V953 | | |
| A0A2V2XR35 | A0A2V2WVL7 | | |
| A0A2V2WX24 | A0A2V2V1M4 | | |

TABLE B

| Chimeric | | Amino acid | Gene bank |
|---|---|---|---|
| antigen | Sequence name | range | sequence ID |
| IBMP-8.1 | Trans-sialidase | 747-774 | XP_820062.1 |
| | 60 S ribosomal protein L19 | 218-238 | XP_820995.1 |
| | Trans-sialidase | 1435-1449 | XP_813586.1 |
| | Surface antigen 2 (CA-2) | 276-297 | XP_813516.1 |
| IBMP-8.2 | Antigen, partial | 13-73 | ACM47959.1 |
| | Surface antigen 2 (CA-2) | 166-220 | XP_818927.1 |
| | Calpain cysteine peptidase | 31-97 | XP_804989.1 |
| IBMP-8.3 | Trans-sialidase | 710-754 | XP_813237.1 |
| | Flagellar repetitive antigen | 15-56 | AAA30177.1 |
| | protein | | |
| | 60 S ribosomal protein L19 | 236-284 | XP_808122.1 |
| | Surface antigen 2 (CA-2) | 279-315 | XP_813516.1 |
| IBMP-8.4 | Shed-acute-phase-antigen | 681-704 | CAA40511.1 |
| | Kinetoplastid membrane | 76-92 | XP_810488.1 |
| | protein KMP-11 | | |
| | Trans-sialidase | 1436-1449 | XP_813586.1 |
| | Flagellar repetitive antigen | 20-47 | AAA30177.1 |
| | protein | | |
| | Trans-sialidase | 740-759 | XP_820062.1 |
| | Surface antigen 2 (CA-2) | 276-298 | XP_813516.1 |
| | Flagellar repetitive antigen | 1-68 | AAA30197.1 |
| | protein | | |
| | 60 S ribosomal protein L19 | 218-238 | XP_820995.1 |
| | Microtubule-associated | 421-458 | XP_809567.1 |
| | protein | | |

Table B includes intact proteins and the specified amino acid segments.

Representative and non-limiting Leishmania antigens include rK39 (UniProt number AOABB9PFHO), rK28, rKR05, rK18, K(39, K28, KR95, and K18/

Representative and non-limiting Dirofilaria immitis include D1T33 SEQ ID NO:6 I GenBank accession no. Q27384.1), and PDi33, and OV33.

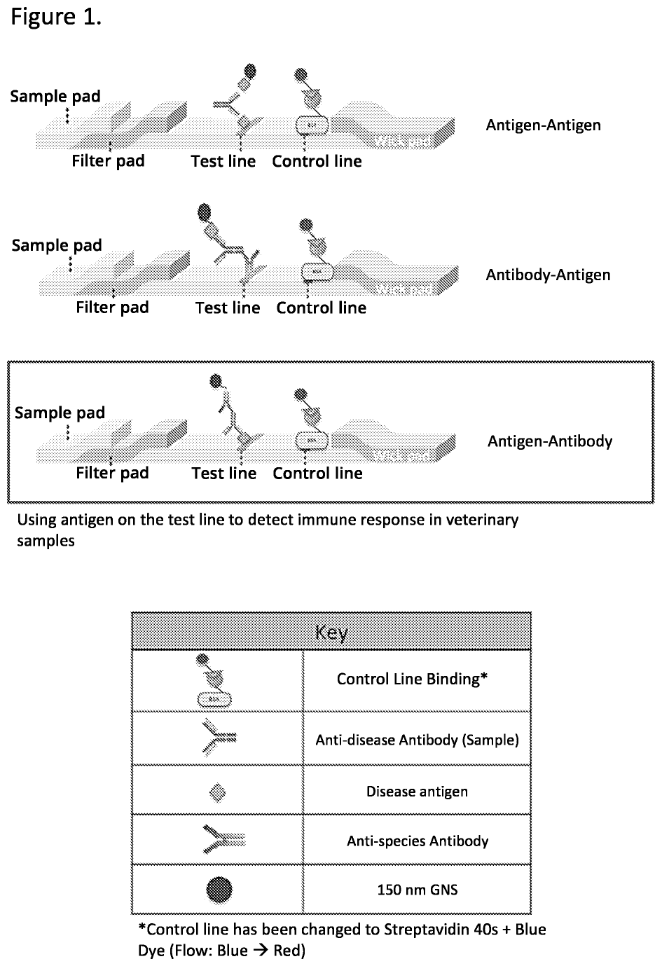

With respect to the presently provided tests, and as will be recognized by those skilled in the art, a variety of antigen and antibody configurations are available for lateral flow testing. FIG. 1 provides three examples wherein, in the top drawing, an Antigen-Antigen configuration is shown. In this configuration, the antigen is attached to a substrate on a test line (and a control antigen is present on a control line, as also shown in the middle and bottom drawings). Antibodies, if present in a biological sample applied to the sample pad and follow through the substrate, bind to the antigen. A detectably labeled antigen is then applied, and a signal from the detectably antigen can be visualized if antibodies to the antigen are present in the sample. In the middle drawing, an Antibody-Antigen configuration is shown. In this configuration, an Ig capture antibody is attached to the substrate. Antibodies to an antigen, if present in the sample, bind to the Ig capture antibody. A detectably labeled antigen is then applied, and a signal from the detectably antigen can be visualized if antibodies to the antigen are present in the sample. In the bottom configuration, an Antigen-Antibody configuration is shown. In this configuration, the antigen is attached to the substrate. Antibodies to an antigen, if present in the sample, bind to the antigen. Detectably labeled Ig antibodies are then applied. If antibodies to the antigen are present in the sample, their presence can be visualized by binding of the detectably labeled antibodies.

In the present disclosure, it was unexpectedly determined that only the bottom configuration of FIG. 1 provides sufficient sensitivity to determine the presence or absence of the antibodies. In particular, for the Chagas test as described further below under the Chagas Example, representative Trypanosoma cruzi 1F8, a recombinant antigen that comprises the epitope of the flagellar calcium-binding protein of Trypanosoma cruzi, and LA204, as described further herein, were used as the antigens attached to the substrate. Secondary antibodies, also referred to as detection antibodies because they are detectably labeled, were selected and tested for their capability to recognize four antibody types: Canine IgG, Canine IgM, Feline IgG, and Feline IgM, which are typically the predominant antibody isotypes found in animal samples. The secondary antibodies were Goat antibodies, obtained from Bethyl Laboratories. All samples that were Chagas positive and negative were confirmed as such by IFA prior to conducting the test depicted test. Using the described reagents, three orientations were evaluated for anti-Chagas antibody detection, as shown in FIG. 1. Antigen-antigen, where either 1F8 or LA204 was fixed on a nitrocellulose membrane and also conjugated a gold nanoshell; antibody-antigen, where one of the four secondary antibodies was fixed on the nitrocellulose membrane and either 1F8 or LA204 was conjugated to the nanoparticle; and lastly, antigen-antibody, where either 1F8 or LA204 was fixed to the nitrocellulose and one of the four secondary antibodies was conjugated to the gold nanoparticle. These orientations were tested with a negative and positive sample, and it was identified that the only orientation that showed sufficient sensitivity contained LA204 on the nitrocellulose membrane and the secondary antibody on the gold nanoshell, i.e., the antigen-antibody configuration as shown in the bottom drawing of FIG. 1. Further, it was unexpectedly determined that the Goat anti-Cat IgG and Goat anti-Cat IgM antibodies were successful at detecting both canine and feline antibodies present in the sample, removing the need for the Goat anti-Dog secondary antibodies in the assay. Thus, in certain embodiments, an immunoassay of this disclosure may employ only anti-Cat IgG and Goat anti-Cat IgM antibodies as detection antibodies in a Chagas test, and can be used to determine antibodies from both feline and canine samples. Additionally, based at least in part on the described results obtained using LA204, it is expected that combining LA204 with additional antigens described herein, including but not necessarily one or more IBMP-8 proteins and/or 1F8, will improve sensitivity of the described Chagas assay. Also encompassed by the present disclosure are use of all combinations of at least 2 proteins in the LA204 antigen mixture.

It will be recognized from the foregoing that, in certain embodiments, a rapid detection immunoassay system and method is provided for qualitatively detecting the presence or absence of antibodies against a target parasite selected from the group consisting of Trypanosoma cruzi, a Leishmania species, and Dirofilaria immitis. Each of these parasites causes a disease condition in a human or animal, with Trypanosoma cruzi being the parasite causing Chagas disease, a Leishmania parasite being the parasite that cause Leishmaniasis, and Dirofilaria immitis being the parasite that causes Heartworm. In an exemplary embodiment of the invention, a test strip is provided which has a plurality of parallel lanes upon which one or more antigens from a specific parasite can be housed. The antigens may be coated onto the test strip in a number of ways that would be well known to those skilled in the art. These antigens are placed on the strip in a manner that so that the different lanes will contain antigens from one parasite, and each lane with antigens will be different from other lanes containing antigens. In one embodiment, the antigens are prepared in the form of an antigenic composition having subset of antigens with antigens specific to one of the parasites referred to above.

In an exemplary method in accordance with the invention, a sample of bodily fluid or other biological matrix from a human or animal patient may be obtained in order to be tested. Such bodily fluid or other biological matrix may be any suitable sample that is readily obtained from a subject, such as during a visit to a doctor or other clinician. Such samples may include blood, serum, saliva, mucus, urine, and other bodily fluids or biological matrices. By biological matrix is meant any substance in the human or animal body that fills a space, e.g., bodily fluids, and such matrices can also include other biological matrices such as an extracellular matrix, a bone matrix, a cartilage matrix, a mitochondrial matrix, a nuclear matrix, and a Golgi matrix. The bodily fluid or other biological matrix sample is then applied to the lanes of the test strip so that the any antibodies in the sample can interact with the antigens on the test strip. By contacting the multiple lanes of the test strip, tests can be carried out to determine the presence or absence of antibodies to multiple parasites at one time. After the sample has had sufficient contact with the test strip so that any antibodies from the sample can interact with antigens on the test strip, the presence or absence of antibodies to one or more target parasites are detected by detecting immunobinding between the antigens on the test strip and the antibodies in the sample. The system detecting the presence of immunobinding can be accomplished in a number of suitable ways that would be recognized by one skilled in the art, including use of markers, signals, or other reagents such as colloidal gold, fluorescent markers, radioactive and non-radioactive isotopes and markers, immunoprecipitation reagents, and other suitable detection materials for qualitatively determining the presence or absence of the target material such as an antibody. This multi-target approach capitalizes in the use of several different antigens for each parasite in order to build-in redundancy and ensure higher sensitivity and specificity of the test.

By virtue of the invention, a system and method is thus provided for qualitatively determining the presence or absence of a plurality of diseases caused by one or more of the target parasites identified above.

In one embodiment, a method is provided wherein a test strip is provided having parallel lanes coated with an antigenic composition comprising antigens from said target parasite, wherein said antigenic composition is provided in separate antigenic composition subsets wherein each subset contains only antigens from a specific target parasite, wherein each subset contains antigens from a different target parasite, and wherein each subset is coated onto one of said parallel lane on said test strip so that a plurality of tests can be carried out at one time. The method can be carried out by obtaining a sample of a bodily fluid or other biological matrix from a human or animal subject, applying said sample to said lanes of said test strip; and determining the presence or absence of antibodies against the target parasite by detecting immunobinding of antigens within each subset to antibodies against said target parasites.

In exemplary embodiments, the antigenic compositions of the invention are based on specific antigens from the target parasites designed to provide highly accurate and sensitive tests that can identify antibodies to the target parasites in a rapid manner, such as during a veterinary or doctor visit, and do so in a manner that reduces false positive and false negative results. The selection of these particular antigens allows for greater precision in the qualitative testing than previously possible, and the present method and also allows for tests of multiple infections at one time.

In this regard, antigen compositions and subsets are provided. As also discussed herein, for Trypanosoma cruzi, these antigens may be selected from the group consisting of 1F8, rTc24 (PGR24-His), TcF, SAPA, TSSA, SAPAITSSA, SAPA/TSSA, FRA, B13, MACH (PEP2, TcD, TcE, SAPA), PEP2, TcD, and TcE, LA204, and an IBMP-8 protein. For a Leishmania parasite, these antigens may be selected from the group consisting of rK39, rK28, rKR95, rK18, K39, K28, KR95, and K18; and for Dirofilaria immitis, these may be selected from the group consisting of ChimDiT33, DIT33, DIT331PDi33, and OV33.

In an exemplary embodiment, the antigens from Trypanosoma cruzi can comprise 1F8: epitope of the flagellar calcium-binding protein (natural or recombinant), Tc24 (PGR24-His): fusion protein found across different evolutionary stages of T. cruzi (natural or recombinant), and the TcF antigen containing tandem sequences of different T. cruzi-specific peptides (natural or recombinant), and LA204. For Leishmania species parasites, the antigen may be rK39:-kinesin-like protein highly conserved among viscerotropic Leishmania species (recombinant), or non-recombinant K39. The protein K39 is a repetitive immunodominant epitope in a kinesin-related protein that is highly conserved among viscerotropic Leishmania species and thus can be sensitive and specific for the diagnosis of Leishmaniasis in humans and animals, such as dogs. In Dirofilaria immitis, the antigen may be DIT33:-pepsin inhibitor (recombinant or non-recombinant). Alternatively other antigens can be added.

In some embodiments, a combination of antigens is provided including antigens from one or more of the above target parasites, and these combinations maximize testing sensitivity and accuracy. The following combinations may be used in the above system and method, wherein each of the recited combinations may also comprise one or more LA204 and/or one or more IBMP-8 proteins:
a. rK39, SAPAITSSA, and ChimDiT33;
b. Tc24 (PGR24-His), SAPA, SAPA/TSSA, FRA, B13, MACH (PEP2, TcD, TcE, SAPA), LA204, IBMP-8, and DIT33; and
c. 1F8, LA204, IBMP-8, Tc24, TcF, SAPA, SAPATSSA, RK39, RK28, RKR95, RK18 and DIT33.

In other embodiments of the invention, a range of antigens that are used in the antigenic composition may be any suitable number that will provide a qualitative determination of the presence of antibodies against said antigen. Such totals, for example, may be between 1 and 100 antigens for each target parasite, 5 to 50 antigens, or 10 to 20 antigens used in each lane of the test strip. The antigens used in accordance with the invention described herein may be in solutions designed to promote binding of the antigens to antibodies from the sample of bodily fluid or other biological matrix. Such solutions and reagents are well known in immunoassay testing, and the antigens in the testing may be employed in appropriate concentrations and volumes, e.g., 25 to 50 μL, with additional examples being provided below.

In an exemplary embodiment, a rapid detection immunoassay method is provided for qualitatively detecting the presence or absence of antibodies against a target parasite selected from the group consisting of Trypanosoma cruzi, a Leishmania parasite, and Dirofilaria immitis in a bodily fluid or other biological matrix from a human or animal subject, said method comprising (a) providing an antigenic composition comprising antigens from a plurality of said target parasites; (b) separating said antigenic composition to form a plurality of antigenic composition subsets, wherein each subset contains only antigens from a single target parasite, and wherein each subset contains antigens from a different target parasite; (c) transferring each of said subsets onto test strips, said test strips having a plurality of parallel lanes, said transferring conducted so as to generate a test strip having separate lanes for each antigenic composition subset so that said test strip is capable of performing an immunoassay of a plurality of a target parasites at the same time; (d) obtaining a sample of a bodily fluid or other biological matrix from a human or animal subject; (e) applying said sample to said lanes of said test strip; and determining the presence or absence of antibodies against the target parasite by detecting immunobinding of antigens within each subset to antibodies against said target parasites.

In accordance with the disclosure, the antigens used in the herein system and method can be any antigenic components that are recognized by antibodies to the target parasites as set forth above. These antigens may be selected from the group consisting of proteins, protein fragments and epitopes, glycans, and peptides. The antigen may also be a blood stage (soluble) or viscerotropic/cardiotropic (tissue-bound) antigen.

In an exemplary embodiment of the invention, the test strips can comprise a nitrocellulose membrane and can be prepared and aligned by establishing a reference line for orientation on a blotter before said antigens are applied to said nitrocellulose membranes. In the above method, separation of the antigenic compositions into the plurality of antigenic composition subsets may be carried out by electrophoresis under denaturing conditions.

With regard to the antigens of the invention as described above, these will include antigens that have had their amino acid chain sequenced. Examples of these antigen sequences include sequences for rK39 (SEQ ID NO:1), SARA (SEQ ID NO:2), TSSA (SEQ ID NO:3), FRA (SEQ ID NO:4), B13 (SEQ ID NO:5), and DIT33 (SEQ ID NO:6).

In another embodiment of the invention, a rapid detection immunoassay method of qualitatively detecting the presence or absence antibodies against a target parasite selected from the group consisting of T. cruzi, Leishmania species, and Dirofilaria immitis in a bodily fluid or other biological matrix from a human or animal subject is provided which comprises (a) providing an antigenic composition comprising antigens from said target parasite; (b) separating said antigenic composition to form separate antigenic composition subsets by electrophoresis, wherein each subset contains only antigens from a specific target parasite, and wherein each subset contains antigens from a different target parasite; (c) transferring said antigenic composition onto nitrocellulose membrane strips in parallel lanes to generate membranes pre-coated with said antigenic composition so that each lane contains an antigenic composition subset; (d) obtaining a sample of a bodily fluid or other biological matrix from a human or animal subject; (e) applying said sample to said lanes of said nitrocellulose membrane strip; and (f) determining the presence of antibodies against the target parasite by detecting immunobinding of antigens within each subset to antibodies against said target parasites. The antigenic composition may include antigens from Trypanosoma cruzi selected from the group consisting of 1F8, rTc24 (PGR24-His), TcF, SAPA, TSSA, SAPA/TSSA, SAPA/TSSA, FRA, B13, MACH (PEP2, TcD, TcE, SAPA), PEP2, TcD, TcE, LA204, and IBMP-8 proteins; antigens from a Leishmania parasite selected from the group consisting of rK39, rK28, rKR95, rK18, K39, K28, KR95, and K18; and antigens from Dirofilaria immitis selected from the group consisting of ChimDiT33, DIT33, DIT33/PDi33, and OV33.

In yet another embodiment of the present method, a rapid qualitative detection immunoassay method is provided for detecting the presence or absence of antibodies against a target parasite selected from the group consisting of T. cruzi, Leishmania parasites, and Dirofilaria immitis in a bodily fluid or other biological matrix from a human or animal subject, said method comprising: (a) providing a test strip having parallel lanes coated with an antigenic composition comprising antigens from said target parasite, wherein said antigenic composition is provided in separate antigenic composition subsets wherein each subset contains only antigens from a specific target parasite, wherein each subset contains antigens from a different target parasite, and wherein each subset is coated onto one of said parallel lane on said test strip so that a plurality of tests can be carried out at one time; (b) obtaining a sample of a bodily fluid or other biological matrix from a human or animal subject; (c) applying said sample to said lanes of said test strip; and (d) determining the presence or absence of antibodies against the target parasite by detecting immunobinding of antigens within each subset to antibodies against said target parasites.

In another exemplary embodiment., a system is provided for a rapid detection immunoassay that qualitatively detects the presence or absence of antibodies against a target parasite selected from the group consisting of Trypanosoma cruzi, a Leishmania parasite and Dirofilaria immitis, as determined from a bodily fluid or other biological matrix from a human or animal subject, said system comprising (a) a test strip having parallel lanes coated with an antigenic composition comprising antigens from said target parasite, wherein said antigenic composition is provided in separate antigenic composition subsets wherein each subset contains only antigens from a specific target parasite, wherein each subset contains antigens from a different target parasite, and wherein each subset is coated onto one of said parallel lane on said test strip so that a plurality of tests can be carried out at one time; (b) a sample of a bodily fluid or other biological matrix from a human or animal subject that can be applied to said test strip; (c) immunobinding detection reagents in each lane of said test strip which provide a signal that indicates immunobinding of the antigens when the sample contains antibodies against the target parasite; and (d) a detection zone on said test strip wherein the signal indicative of immunobinding between the antigens on the test strip and antibodies against said target parasites can be observed. The antigens from Trypanosoma cruzi used in this system may be selected from the group consisting of 1F8, rTc24 (PGR24-His), TcF, SAPA, TSSA, SAPA/TSSA, SAPA/TSSA, FRA, B13, MACH (PEP2, TcD, TcE, SAPA), PEP2, TcD, TcE, LA204 and an IBMP-8 protein; the antigens from a Leishmania parasite may be selected from the group consisting of rK39, rK28, rKR95, rK18, K39, K28, KR95, and K18; and the antigens from Dirofilaria immitis are selected from the group consisting of ChimDiT33, DIT33, DIT33/PDi33, and OV33. Particular combinations of antigens may be used in this system whereby each combination includes at least one antigen from each parasite being tested. Examples of such combinations of antigens may include (a) rK39, SAPA/TSSA, and ChimDiT33; (b) Tc24 (PGR24-His), SAPA, SAPA/TSSA, FRA, B13, MACH (PEP2, TcD, TcE, SAPA) and DIT33; or (cc) 1F8, Tc24, TcF, SAPA, SAPA/TSSA, RK39, RK28, RKR95, RK18 and DIT33. These antigens may be recombinant or non-recombinant, and can be any antigen or antigen fragment capable of binding to an antibody from one of the target parasites, such as proteins, protein fragments or epitopes, glycans, and peptides.

A variety of test strip systems well known in the immunoassay art may be used in the method and system described herein, such as membrane test strips made out of nitrocellulose. The test strips may also be any of a wide range of commercially available test strips that are set up to include a wide variety of test strip reagents and modes of detection. In one system, the immunoassay may comprise a nitrocellulose membrane tests strip that includes a reference line for orientation on a blotter. The test strip will generally contain a number of antigens specific to each of said target parasites, and the antigens will be in suitable numbers so that they will be recognized and will exhibit immunobinding with the respective target parasite antibodies in the bodily fluid or other biological matrix being tested. Such a system is suitable for a wide variety of immunoassay methods and devices including lateral flow immunoassays, multiple antigen blot immunoassays, cross-blot immunoassays, direct or indirect Enzyme-linked immunosorbent assays (ELISA), enzymatic immunoassays, sandwich immunoassays, electrochemilumescence immunoassays, dipstick immunoassays, and radioisotope immunoassays . . . .

As indicated above, the test strip of the invention may comprise any suitable test strip commonly used in immunoassay procedures and apparatuses that would be well known to one skilled in the art. The test strip is used for qualitatively determining the present or absence of antibodies against a target parasite selected from the group consisting of Trypanosoma cruzi, a Leishmania parasite and Dirofilaria immitis in a bodily fluid or other biological matrix from a human or animal subject, and the test strip will generally include parallel lanes coated with an antigen from each of said target parasites, with the antigens configured so that there is a different antigen specific to a particular target parasite in each lane. The antigens may be provided in the form of an antigenic composition which is separated to form antigenic composition subsets, each of which contains antigens from only one of said target parasites. These antigen subsets may be configured on the test strip so that a particular lane will include antigens from only one of said target parasites, and each lane will contain antigens that are different from each other. In one embodiment, the test strip will have a plurality of lanes wherein one lane will contain antigens only from Trypanosoma cruzi, one lane will include only antigens from a Leishmania parasite, and one lane will contain antigens only to Dirofilaria immitis. Other configurations are possible wherein the test strip will include antigens from fewer than all three of the target parasites, including combinations of any two of the parasites, or a single parasite individually.

In another exemplary embodiment, the test strip contains on or more antigenic subsets rom a specific target parasite, and in the case where multiple target parasites are included, each subset will contains antigens from a different target parasite, and each subset will be coated onto one of said lanes on said test strip so that a plurality of tests can be carried out at one time. As indicated herein, the test strips of the invention may contain antigens from Trypanosoma cruzi selected from the group consisting of 1F8, rTc24 (PGR24-His), TcF, SAPA, TSSA, SAPA/TSSA, SAPA/TSSA, FRA, B13, MACH (PEP2, TcD, TcE, SAPA), PEP2, TcD, TcE, LA204, and an IBMP-8 protein; antigens from a Leishmania parasite selected from the group consisting of rK39, rK28, rKR95, rK18, K39, K28, KR95, and K18; and/or antigens from Dirofilaria immitis selected from the group consisting of ChimDiT33, DIT33, DIT33/PDi33, and OV33. In specific combinations of antigens from the various target parasites, such combination of antigens may include (a) rK39, SAPA/TSSA, and ChimDiT33; (b) Tc24 (PGR24-His), SAPA, SAPA/TSSA, FRA, B13, MACH (PEP2, TcD, TcE, SAPA) and DIT33; and (c) 1F8, LA204, Tc24, an IBMP-8 protein, TcF, SAPA, SAPA/TSSA, RK39, RK28, RKR95, RK18 and DIT33. Such antigens may also be one or more of the specific sequences SEQ ID NOs:1-6 as included herein.

In another embodiment, a rapid detection immunoassay test kit for qualitatively detecting the presence or absence of antibodies against a target parasite selected from the group consisting of T. cruzi, Leishmania species and Dirofilaria immitis in a bodily fluid or other biological matrix from a human or animal subject, wherein the test kit comprises (a) a test strip having parallel lanes coated with an antigenic composition comprising antigens from said target parasite, wherein said antigenic composition is provided in separate antigenic composition subsets wherein each subset contains only antigens from a specific target parasite, wherein each subset contains antigens from a different target parasite, and wherein each subset is coated onto one of said parallel lane on said test strip so that a plurality of tests can be carried out at one time; (b) immunobinding detection reagents in each lane of said test strip which provide a signal that indicates immunobinding of the antigens when the sample contains antibodies against the target parasite; and (c) a detection zone on said test strip wherein the signal indicative of immunobinding between the antigens on the test strip and antibodies against said target parasites can be observed.

In another embodiment, a method is provided for diagnosing an infection caused by a parasite selected from the group consisting of T. cruzi, Leishmania parasites, and Dirofilaria immitis in a bodily fluid or other biological matrix from a human or animal subject, said method comprising: (a) providing a test strip having parallel lanes coated with an antigenic composition comprising antigens from said target parasite, wherein said antigenic composition is provided in separate antigenic composition subsets wherein each subset contains only antigens from a specific target parasite, wherein each subset contains antigens from a different target parasite, and wherein each subset is coated onto one of said parallel lane on said test strip so that a plurality of tests can be carried out at one time; (b) obtaining a sample of a bodily fluid or other biological matrix from a human or animal subject; (c) applying said sample to said lanes of said test strip; and (d) diagnosing the infection by determining the presence or absence of antibodies against the target parasite by detecting immunobinding of antigens within each subset to antibodies against said target parasites.

Still a further embodiment of the invention is a method of determining whether a human or animal subject has a disease condition selected from the group of Chagas disease, Leishmaniasis, and Heartworm, said method comprising: (a) providing a test strip having parallel lanes coated with an antigenic composition comprising antigens from parasites that cause said disease conditions, wherein said antigenic composition is provided in separate antigenic composition subsets wherein each subset contains only antigens from a specific parasite causing one of said disease condition, wherein each subset contains antigens from only said specific parasite, and wherein each subset is coated onto one of said parallel lane on said test strip so that a plurality of tests can be carried out at one time; (b) obtaining a sample of a bodily fluid or other biological matrix from a human or animal subject; (c) applying said sample to said lanes of said test strip; and (d) determining whether the human or animal subject has a disease condition selected from the group of Chagas disease, Leishmaniasis, and Heartworm by detecting the presence or absence of antibodies against said specific parasites causing said disease conditions, said presence or absence determined by detecting immunobinding of antigens within each subset to antibodies against said specific parasites.

In another embodiment, a rapid detection immunoassay method is provided for qualitatively detecting the presence or absence of antibodies against a target parasite selected from the group consisting of T. cruzi, a Leishmania parasite, and Dirofilaria immitis in a bodily fluid or other biological matrix from a human or animal subject, said method comprising: (a) providing a test strip comprising a plurality of lanes, wherein each lane in said plurality of lanes contains an antigen from a specific target parasite, and each of said lanes contains an antigen from a different target parasite; (b) obtaining a sample of a bodily fluid or other biological matrix from a human or animal subject; (c) applying said sample to the plurality of lanes of said test strip; and (d) determining the presence of antibodies against the target parasite by detecting immunobinding of antigens within each subset to antibodies against said target parasites; Also provided is a rapid detection immunoassay for qualitatively detecting the presence or absence of antibodies against a target parasite selected from the group consisting of Trypanosoma cruzi, a Leishmania parasite, and Dirofilaria immitis in a bodily fluid or other biological matrix from a human or animal subject, said immunoassay comprising: (a) a test strip comprising a plurality of lanes, wherein each lane in said plurality of lanes contains an antigen from a specific target parasite, and each of said lanes contains an antigen from a different target parasite; and (b) a sample of a bodily fluid or other biological matrix from a human or animal subject that can be applied to said test strip so that the presence of antibodies against the target parasite can be determined by virtue of immunobinding between said antigens and said antibodies; wherein the antigen from Trypanosoma cruzi is selected from the group consisting of 1F8, rTc24 (PGR24-His), TcF, SAPA, TSSA, SAPA/TSSA, SAPA/TSSA, FRA, B13, MACH (PEP2, TcD, TcE, SAPA), PEP2, TcD, TcE, LA204 and an IMBP-protein, wherein the antigen from a Leishmania species is selected from the group consisting of rK39, rK28, rKR95, rK18, K39, K28, KR95, and K18; and wherein the antigen from Dirofilaria immitis is selected from the group consisting of ChimDiT33, DIT33, DIT33/PDi33, and OV33.

In certain embodiments of the present detection method is carried out using a Lateral flow assay (LFA) which provides multiple diagnostic assays for various parasites of veterinary/human concern. The method of detection using the antigenic composition of the present invention is normally carried out by conducting in vitro assay using the antigens of the invention for the detection of one or more of the target parasitic pathogens as described herein, and the assays can also be repurposed for other immunoassay methods as needed. Given the multifaceted life cycles of the parasites Trypanosoma cruzi, Leishmania species pathogens, and Dirofilaria immitis and the occurrence of multiple developmental stages with different and complex antigenic makeups for each stage, the present invention involves selecting a specific antigen array comprising a mixture of at least one recombinant antigen known to be present through all stages of infection for each parasite in order to increase yield of detection, particularly in pre-patent and latent stages of infection.

As a result, in accordance with the invention, an immunoreactive antigenic composition suitable for detecting the different life stages of Trypanosoma cruzi, Leishmania species such as Leishmania infantum, and Dirofilaria immitis, is provided which includes the combination of the antigen Tc24 from Trypanosoma cruzi, of the antigen K39 from a Leishmania parasite, and DIT33 from Dirofilaria immitis. These antigens are thus useful in identifying and diagnosing the diseases caused by these parasites. For example, Chagas disease, otherwise known as American trypanosomiasis, may be diagnosed using one or more antigens to Trypanosoma cruzi such as Tc24 which can be utilized as a pan-stage marker in the present antigen repertoire. Tc24 is a 24-kDA protein of the T. cruzi parasite that is found across all developmental stages of the parasite, specifically in the flagella of trypomastigotes (infectious stage) and epimastigotes (vector—bound stage). With regard to the disease visceral leishmaniasis, one of the antigens used in accordance with the invention is K39, a large kinesin-related protein expressed predominantly by amastigotes (intracellular form of the parasite in the mammalian host). With regard to Heartworm infection, the invention may include the antigen DIT33/PDi33 from Dirofilaria immitis, a homologue of the aspartyl protease inhibitor (0v33) of the related parasite Onchocerca volvulus. DIT33 is also a pan-stage marker present across all stages of the parasite in the mammalian host (microfilariae, L3, L4, adult males, and females). As a result of the fact that certain antigens are found across all life stages of these parasites makes the antigens as described herein particularly useful in identifying antibodies to the parasites at multiple life stages of the parasite life cycle.

The antigens used in the present invention can be obtained from a variety of sources, including well known commercially available sources where these antigens may be purchased. Alternatively, it is also possible to generate the antigens used in the invention by culturing transformed cells with an expression vector (e.g., recombinant DNA or other nucleic acid) encoding for each and every one of the antigens provided herein. These generation methods allow for the expression and purification of individual antigens so as to then generate the antigen composition that will be used in the immunoassay methods, systems, and devices of the present invention as described herein.

For example, there are several commercially available sources of the antigens of the present invention such as reflected below:
K39: https://www.rekombiotech.com/en/antigens/humans/k39
r1F8: https://www.creative-diagnostics.com/Recombinant-Trypanosoma-Cruzi-1F8-25-8-kDa-His-tagged-4315-407.htm
TcF: https://www.creative-diagnostics.com/Active-T-cruzi-Chagas-TcF-protein-176190-407.htm
DIT33: https://www.creative-biolabs.com/vaccine/recombinant-canine-heartworm-Dirofilaria-immitis-dit33-protein-aa-18-234-28438.htm
OV33: https://www.bio-connectnVproteins-signaling-moleculelimmunodominant-antigen-ov33-3-recombinant-protein-onchocerca-volvulus/opca51067/sfid/10496061
https://www.hoelze biotech.com/en/cusabio-proteins-other-csb-ep324156oeg-recombinant-immunodominant-antigen-ov33-3.html

The present disclosure includes the design of a qualitative, membrane based immunoassay for detecting Trypanosoma cruzi, Leishmania species such as Leishmania infantum and Dirofilaria immitis in canine serum and whole-blood matrices by applying various antigens for each parasite in order to increase sensitivity and specificity. As indicated herein, there are many forms of immunoassay that may be utilized in accordance with the invention, and exemplary modalities for carrying out the present diagnostic method would include (1) Lateral flow immunoassay; (2) Multiple antigen blot assay; and (3) Enzyme-linked immunosorbent assay (ELISA).

In one exemplary example, the present immunoassay system and method as described herein may include a first testing modality comprising the use of a membrane, such as a nitrocellulose membrane, that is pre-coated with the antigens (recombinant or non-recombinant) as described herein. These antigens may be arrayed and lined up in different test line regions with separate controls to ensure assay flow and performance. This method also may include an immunoreactive composition of said antigens and a suitable stabilizing and conjugate solution. In an exemplary method, both the bodily fluid or other biological matrix being tested, such as blood serum, and the immunobinding conjugate are placed in a sample pad creating an admixture that migrates by capillary action through the membrane to react with the antigens on the membrane. If antibodies from the target parasites are present as would be detected by immunereactivity to any of the target antigens, the test line will display a signal, such as immunofluorescence of a colorimetric reaction, and this signals the presence of antibodies against the target antigen in the bodily fluid or other biological matrix sample.

A second testing modality may comprise the simultaneous screening of said antigens using a dot immunobinding assay which comprises lining up the antigen repertoire on a nitrocellulose membrane reformatted into a dipstick immunoassay format. It is also possible to include a third modality which would be in proper format for an Enzyme-linked immunosorbent assay (ELISA).

In one exemplary embodiment, a method is provided for qualitative, membrane-based immunobinding assay testing for the detection of antibodies against Trypanosoma cruzi, Leishmania species, and Dirofilaria immitis both in serum and whole blood matrices. In such a method, recombinant antigens and/or peptides obtained from known sequences of Trypanosoma cruzi, Leishmania sp., and Dirofilaria immitis synthetized by various methods are separated by electrophoresis under denaturing conditions and electro transferred or transferred by diffusion onto nitrocellulose membrane strips (using the cross-blot method) in parallel lanes to generate membranes pre-coated with recombinant antigens/peptides. In one such method, nitrocellulose membranes are prepared and aligned by establishing a reference line for orientation on the blotter before being sensitized with the said antigens. In the general case, a range of antigens, such as 1-to-600, 5-to-50, or 10-to-20 antigens can be tested simultaneously on each membrane. Using the composition of specific antigens in accordance with the invention overcomes the disadvantages of previous approaches using single target antigens by providing testing redundancy. In addition, this novel composition (based on an admixture of antigens) targeting multiple parasites of interests is comparable or superior in terms of specificity, sensitivity and reproducibility for detection of Trypanosoma cruzi, Leishmania sp and Dirofilaria immitis, and thus the diseases associated with these parasites, respectively Chagas disease, Leishmaniasis, and Heartworm.

As provided below, the sensitivity and specificity of some of the antigens used in the invention are shown in the charts below:

### Leishmania sp:

| Antigen | Sensitivity | Specificity |
|---|---|---|
| rK39 | 97.3% | 100% |
| rK28 | 95.9% | 100% |
| rKR95 | 95.9% | 100% |
| rK18 | 89.10% | 98.60% |

Trypanosoma cruzi:

| Antigen | Sensitivity | Specificity |
|---|---|---|
| SAPA | 86.80% | 100% |
| SAPA/TSSA | 94.70% | 100% |
| FRA, B13, MACH (PEP2, | 99.60% | 97.50% |
| TcD, TcE, SAPA) | | |

In one embodiment of the above antigens, the antigen combination SAPA/TSSA VI has been shown to have a significant reactivity (95%) in dog sera.

Dirofilaria immitis:

| Antigen | Sensitivity | Specificity |
|---|---|---|
| ChimDiT33 | Not determined | Not determined |

### REFERENCES

Additionally, specific embodiments of the present invention may include compositions that comprise the following combination/admixture of antigens: rK39, SAPA/TSSA, ChimDT33 embracing a single antigen for each parasite.

A further embodiment is a composition comprising: Tc24 (PGR24-His), SAPA, SAPA/TSSA, FRA, B13, MACH (PEP2, TcD, TcE, SAPA), LA204, and a IBMP-8 protein, and DIT33 restricted to the cardiotropic parasites Trypanosoma cruzi and Dirofilaria immitis.

In yet another embodiment the composition consists of the following broad panel of antigens: 1F8, Tc24, TcF, SAPA, SAPA/TSSA, RK39, RK28, RKR95, RK18, DIT33, LA204, and an IBMP-8 protein.

In another exemplary embodiment, the immunoassay may be a cross-blot system (apparatus) that is built for commercial scale and which is used for delivery of an antigen preparation (membrane sensitizationincubation). A series of effective antigenic concentrations may be prepared to ensure the antigens will bind with antibodies from said target parasites, with such concentrations ranging from 10 to 100 uL, or amounts within this range such as 20 to 80 UI or 25 to 50 uL, of each antigenic preparation can be transferred (blotted) onto the membrane and incubated for a suitable period, e.g., 30-90 minutes or 45-60 minutes followed by washing by a suitable wash solution, such as PBS-0.05%, Tween 200, and blocking with a suitable blocking solution (such as 5% non-fat milk in PBS-0.05% Tween 20) for a suitable period of time, e.g., 1-3 hours, or approximately 2 hours. The membranes may subsequently be washed by a suitable immunoassay wash solution (e.g., PBS-0.05% Tween-20) for a suitable time (e.g., ×3 for 10 minutes) and incubated for suitable conjugation period, e.g., 30 to 90 minutes, or about 60 minutes, using standard conjugation methods and revealed to assess reactivity of the assayed sera.

For use in lateral flow, dipstick-based diagnostic format a blend of stable liquid conjugate along with the tested sera will react with the said composition of antigens which will appear as positive test lines for the specific parasite(s) present, shortening the time to 15 to 25 minutes total test time.

Another aspect of this disclosure is that this composition of antigens can be used to improve any format of detection immunoassays for the detection of anti-T. cruzi, anti-Leishmania sp and anti-Dirofilaria independently of the method of detection (enzyme immunoassay, electrochemilumescence assay or radioisotope) as well as the format for testing (ELISA direct/indirect, sandwich, or test strip).

In another aspect of the disclosure, a device is provided for conducting an immunoassay of antibodies against a target parasite selected from the group consisting of T. cruzi, a Leishmania parasite, and Dirofilaria immitis in a bodily fluid or other biological matrix from a human or animal subject, said device comprising a nitrocellulose membrane having strips in parallel lanes that are pre-coated with an antigenic composition comprising two or more antigens as described herein; wherein each of said lanes contains an antigenic composition exclusively obtained from one of said target parasites, and provides a surface for receiving a sample of bodily fluid or other biological matrix from a human or animal subject; and wherein said nitrocellulose membrane further comprises detection reagents that detect binding between an antibody to a target parasite located in said bodily fluid or other biological matrix sample so as to qualitatively detect antibodies to the target parasite in said sample when said binding is detected. The device may include detection reagents or other detection materials that generate a signal when binding of the antigen to an antibody is detected. The signal is selected from the group consisting of a colorimetric signal, a fluorescent signal, a chemiluminescent signal, a radioactive signal, and a visual signal.

In yet another exemplary method, an antigenic composition is provided comprising recombinant antigens, peptides, or mixtures thereof obtained from known sequences of T. cruzi, Leishmania sp and Dirofilaria immitis. This antigenic composition may be separated by electrophoresis under denaturing conditions and electrotransferred or transferred through other suitable means, wherein said antigenic composition is transferred onto nitrocellulose membrane strips in parallel lanes to generate membranes pre-coated with said antigenic composition so that each lane contains an antigenic composition exclusively comprised of recombinant antigens, peptides, or mixtures thereof from a single target parasite. The testing may be conducted by obtaining a sample of a bodily fluid or other biological matrix from a human or animal subject, such as a dog or other pet susceptible to one or more of the target parasites, and applying the sample to the lanes of said nitrocellulose membrane strip, and the presence or absence of antibodies to the target parasite in the bodily fluid or other biological matrix sample is determined by detecting the immunoassay binding of the antigens in the antigenic composition with immunobinding antibodies from said target parasite which reflect exposure to the human or animal subject to one of the target parasites and the diseases associated with the respective parasites.

### EXAMPLES

The following Examples are intended to illustrate but not limit the disclosure. In assays described in these examples, detectably labeled goat serum may be 5%-10% of the volume of a mixture that is applied to a test strip. In embodiments, the sample is allowed to contact the test line antigen prior to application of the labeled detection antibodies. In embodiments, a NaCl concentration in which goat serum is used is more than 1.2M. In embodiments, the NaCl concentration in goat serum is 2.4M, or at least 2.4M. Other components used in the assays can comprise MgCl2, dextran, and sodium citrate.

### Chagas Example

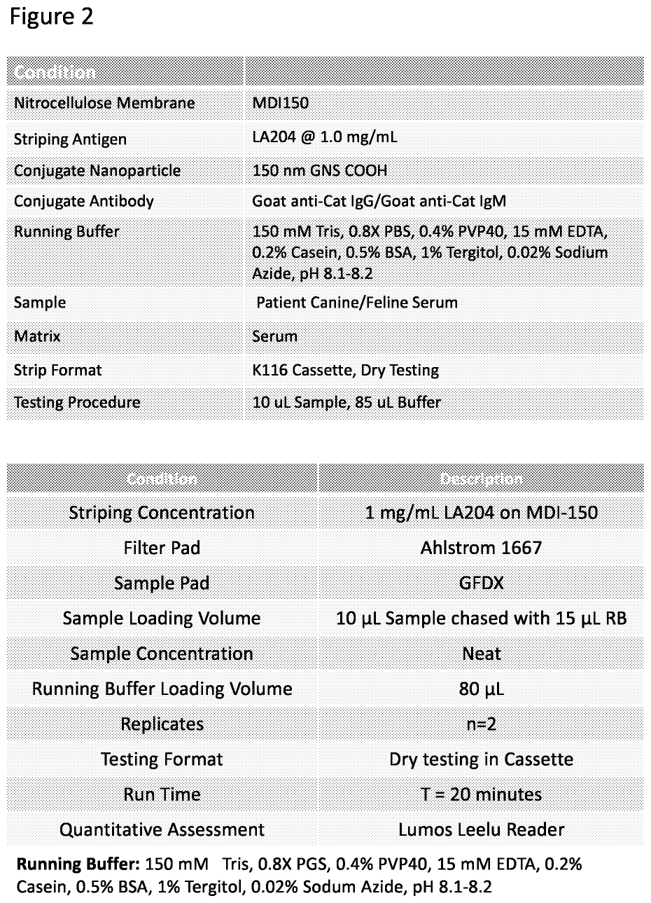

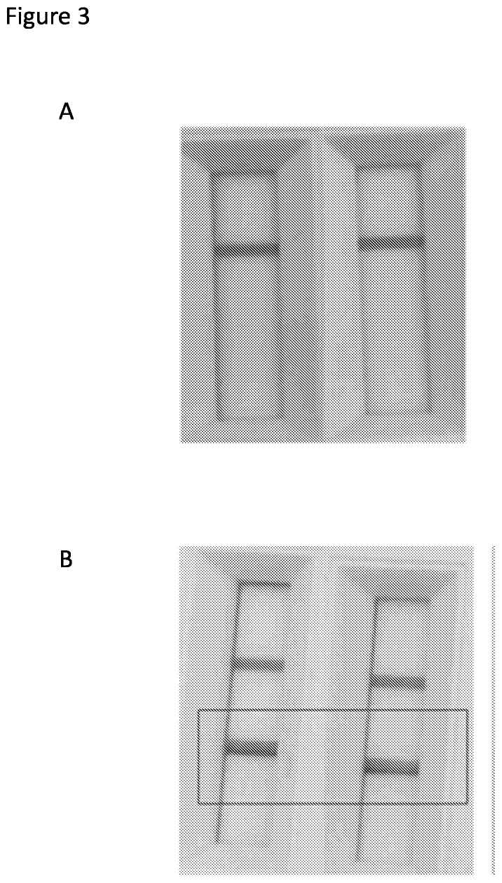

For Chagas, the antigen attached to the substrate was LA204 at 1.0 mg/mL. The substrate was a MD1150 nitrocellulose membrane. The running buffer was 150 mM Tris, 0.8×PBS, 0.4% PVP40, 15 mM EDTA, 0.2% Casein, 0.5% BSA, 1% Tergitol, 0.02% Sodium Azidein a 80 μL volume. The sample loading was 10 μL sample chased with 15 μL RB. The reading window is about 37-53 mm within a 25 mm segment of the strip. The nitrocellulose membrane is flanked by a buffer pad, a sample pad, and a filter pad on the sample loading side, and a wick pad at the end of the flow direction. The entire length of the strip is 80 mm. A mixture of Goat anti-Cat IgG/Goat anti-Cat IgM serum was used. The conjugate nanoparticle on the detection antibodies is 150 nm GNS COOH. The control is Streptavidin 40s conjugated goat serum and Blue Dye; biotinylated BSA is attached to the substrate. The sample contained canine or feline serum. The sample comprised 10 uL Sample, 85 uL Buffer. The general sample conditions are shown in FIG. 2. All negative samples and positive samples were confirmed to be Chagas negative or positive, respectively, by IFA prior to the lateral flow strip tests. A representative result for a Chagas negative sample is shown in FIG. 3, panel A. A representative result for a Chagas positive sample is shown in FIG. 3, panel B.

Positive and negative results consistent with the IFA-predetermined status for Chagas were obtained for both canine and feline samples using the Goat anti-Cat IgG/Goat anti-Cat IgM. Thus, this Example demonstrates that use of Goat anti-Cat IgG/Goat anti-Cat IgM is sufficient for determining positive and negative results for both canine and feline subjects. Sensitivity of 50% was achieved using 18 positive and 12 negative samples. The disclosure includes increasing sensitivity of this assay by combining the LA204 antigens with at least one additional antigen, such as an IBMP-8 antigen, or a 1F8 antigen. The disclosure also includes increasing sensitivity by combining least one of an IBMP-8 protein with at least one of 1F8, Tc24, SAPA, B13, PEP2, FRA, TSSA, TcD, TcF, or TcE.

### Heartworm Example

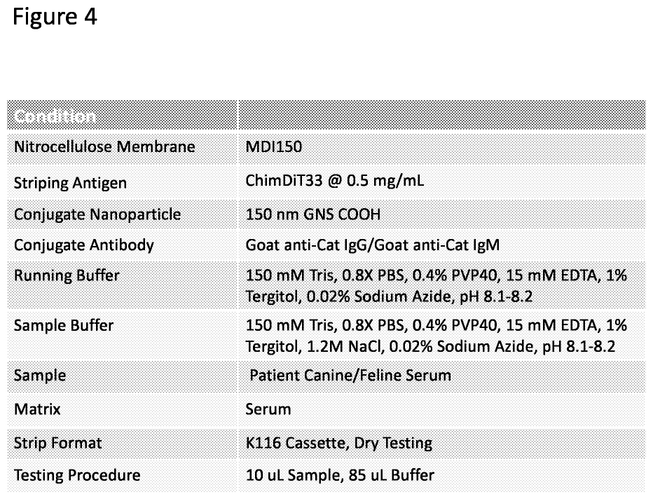

For heartworm, conditions were similar to the Chagas assay. 0.5 mg/mL Dit33 was applied to the strip at 0.08 uL/mm. The running buffer was 150 mM Tris, 0.8×PBS, 0.4% PVP40, 15 mM EDTA, 1% Tergitol, 0.02% Sodium Azide, pH 8.1-8.2. The sample buffer was 150 mM Tris, 0.8×PBS, 0.4% PVP40, 15 mM EDTA, 1% Tergitol, 1.2M NaCl, 0.02% Sodium Azide, pH 8.1-8.2. 10 uL Sample in 85 uL Buffer was used. A representative table of assay components is shown in FIG. 4. Positive and negative results were obtained from feline and canine samples.

### Leishmania Example

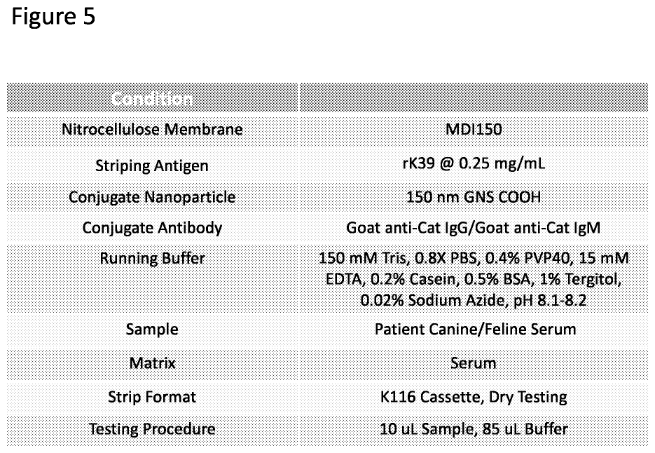

For Leishmania, general conditions were similar to heartworm. The antigen was rK39 at 0.25 mg/mL. The running buffer comprised 150 mM Tris, 0.8×PBS, 0.4% PVP40, 15 mM EDTA, 0.2% Casein, 0.5% BSA, 1% Tergitol, 0.02% Sodium Azide, pH 8.1-8.2. Positive and negative results were obtained from feline and canine samples. A representative table showing Leishmania assay conditions is provided in FIG. 5, wherein the sample buffer comprises 2.4M NaCl. Positive and negative results were obtained from feline and canine samples.

SEQUENCES:

rK39
>tr|A0A6B9PFH0|A0A6B9PFH0_LEIIN Kinesin-like protein (Fragment) OS = Leishmania
infantum OX = 5671 PE = 4 SV = 1
[SEQ ID NO: 1]
ELATEWEDALRERALAERDEAAAAELDAAASTSQNARESACERLTSLEQQLRDSEER
AAELASQLESTTAAKMSAEQDRESTRATLEQQLRESEERAAELASQLESTTAAKMSAE
QDRESTRATLEQQLRDSEERAAELASQLESTTAAKMSAEQDRESTRATLEQQLRESE
ARAAELAS
SARA
>tr|Q00773|Q00773_TRYCR Shed-acute-phase-antigen OS = Trypanosoma cruzi
OX = 5693 GN = SAPA PE = 4 SV = 1
[SEQ ID NO: 2]
MGRTVVGASRMFWLTHFVPLLLALCPSEPAHALAPGSSRVELFKRQNSTVPFEENGE
VRQRVVHSFRLPALVNVDGVMVAIADARYDTSNDNSLIDTVVKYSVDDGETWETQIAIK
NSRASSVSRVVDPTVIVKGNKIYVLVGSYNSSTSYWTSHGDARDWDILLAVGEVTKSIV
DGKTTANITWGSPVSLKEFFPAYMEGMHTNQFLGGAGVAIVASNGNLVYPVQVTNKR
KQVFSKIFYSEDDGKTWKFGKGRSDFGCSEPVALEWEGKLIINTRVDYRRRLVYESSD
TEKPWVEAVGTLSRCWGPSPKSDQPGSQSSFTAVTIEGMRVMLFTHPLNFKGWWLR
DRLNLWLTDNQRIYNVGQLSIGDENSAYSSVLYKDDKLYCLHEINSNEVYSLVFARLVG
ELRIIKSVLQSWKKWDSHLSSICTPADPAASSSERVCGPAVTTVGLAGFLSHSANKTK
WKDAYRCVDASTANAERVSNGLKFAGVGGGALWPVSQQGQNQRYRFANHAFTLVA
SVTIHEAPSVASPLLGASLDSSGGKKLLGLSYDEKHQWQPIYGSTPVTPTGSWETGKR
YHVVLTMANRNGSVYVDGELLKGSGQTWVPDRTPDISHFYVGGYGRSDMPTISHVTV
NNVLLYNRQLNAEEIKTLFLSQDLIGTEAHMDSSSDSSAHGTPSTPVDSTAHGTPSTPA
DSSAHSTPSTPADSSAHSTPSTPVDSSAHSTPSTPADSSAHSTPSTPADSSAHSTPST
PVDSTAHGTPSTPADSSAHSTPSTPVDSSAHSTPSTPADSSAHSTPSTPVDSSAHSTP
STPADSSAHGTPSTPVDSSAHSTPSTPADSSANGTVLILPDGAALSTFSGGGLLLCAC
ALLLHVFFTAVFF
TSSA
>tr|D0VAW4|D0VAW4_TRYCR Trypomastigote small surface antigen (Fragment)
OS = Trypanosoma cruzi OX = 5693 GN = TSSA PE = 4 SV = 1
[SEQ ID NO: 3]
MTTCRLLCALLALALCCCLXACTTANGGSTXSTPPSGTENKPAXGEAPSQPGASSGEA
EASS
FRA
>tr|Q26921|Q26921_TRYCR Flagellar repetitive antigen (FRA) protein (Fragment)
OS = Trypanosoma cruzi OX = 5693 PE = 4 SV = 1
[SEQ ID NO: 4]
MEQERRQLLEKDPRRNAKEIAALEESMNARAQELAREKKLADRAFLDQKPERVPLAD
VPLDDDSDFVA
B13
>tr|Q7YZF5|Q7YZF5_TRYCR B13 antigen (Fragment) OS = Trypanosoma cruzi
OX = 5693 PE = 4 SV = 1
[SEQ ID NO: 5]
PPPFGQAAAGDKPSPFGQAAAGDKPPPFGQAAAGDKPSPFGQAAAGDKPPPFGQAA
AGDKPSPFGQAAAGGKPPPFGQAAAGDKPSPFGQAAAGDKPPPFGQAAAGDKPSPF
GQAAAGDKPPPFGQAAAGDKPPPFGQAAEGDKPPPFGQAAAGDKPAPFGQAAEGD
KPPPFGQAAAADKPSLFGQAAAGDKLSLFGQAAAGDKPPPFGQAAEGDKPPPFGQA
AAGD
DIT33
>sp|Q27384|API_DIRIM Pepsin inhibitor Dit33 OS = Dirofilaria immitis
OX = 6287 GN = DIT33 PE = 2 SV = 1
[SEQ ID NO: 6]
MKILFCFVLLAIAALRASVINRHNKRFAGFSVAGIGGTAGCVVVDNKLFANSFYLRDLTT
EEQRELAQYVEDSNQYKEEVKTSLEERRKGWQLARHGEKDAKVLSSLAEKKFPKPPK
KPSFCSAGDTTQYYFDGCMVQNNKIYVGRMYVRDLTSDEINQLKTFDAKMTAYQKYL
SSSIQQQVDSLFGDKSNLFNLFTDTRHETSSQPSDATTISTTTQAPVEPPETPHFCIAIY

What is claimed is:

1. A combination of materials comprising:
a) a first substrate comprising at least one Trypanosoma cruzi antigen attached to the first substrate; and
b) detectably labeled antibodies that are capable of specifically binding to antibodies produced by an animal that has been exposed to Trypanosoma cruzi.

2. The combination of materials of claim 1, wherein the at least one Trypanosoma cruzi antigen comprises at least one of a Trypanosoma cruzi antigen that is an LA204 protein selected from the proteins in Table A, an IBMP-8 protein selected from the proteins of Table B, 1F8, Tc24, SAPA, B13, PEP2, FRA, TSSA, TcD, TcF, or TcE, or an antigenic fragment of any of said proteins, or a combination of said proteins, or a combination of said antigenic fragments, or a combination of said proteins and antigenic fragments thereof.

3. The combination of materials of claim 2, wherein the at least one Trypanosoma cruzi antigen comprises a combination of LA204 proteins, or antigenic fragments thereof.

4. The combination of materials of claim 3, wherein the at least one Trypanosoma cruzi antigen comprises all of the proteins of LA204 as in Table A.

5. The combination of materials of claim 4, wherein the at least one Trypanosoma cruzi antigen comprises the 1F8 protein, an IBMP-8 protein from Table B, or an antigenic fragment thereof.

6. The combination of materials of claim 4, wherein the at least one Trypanosoma cruzi comprises the 1F8 or the IBMP-8 protein.

7. The combination of materials of claim 1, further comprising:
i) at least one Leishmania protein or antigenic fragment thereof attached to the first substrate or to a second substrate, and detectably labeled antibodies that are capable of specifically binding to antibodies produced by an animal that has been exposed to the Leishmania, wherein said Leishmania protein is optionally rK39, rK28, rKR95, rK18, K39, K28, KR95, and K18; or
ii) at least one Dirofilaria immitis antigen attached to the first substrate, the second substrate, or a third substrate and detectably labeled antibodies that are capable of specifically binding to antibodies produced by an animal that has been exposed to the Dirofilaria immitis; wherein said Dirofilaria immitis antigen is optionally DIT33, ChimDiT33, PDi33, or antigenic fragments thereof;
iii) a combination of i) and ii).

6. The combination of materials of claim 1, wherein the substrate is configured to permit flow of a liquid biological sample through the substrate, and wherein said substrate is optionally nitrocellulose.

7. The combination of materials of claim 1, wherein the detectably labeled antibodies comprise a detectable label that is visually detectable.

8. The combination of materials of claim 7, wherein the detectable label comprises gold particles.

9. The combination of materials of claim 8, wherein the detectably labeled antibodies comprise polyclonal goat anti-feline antibodies.

10. The combination of materials of claim 9, wherein the polyclonal goat anti-feline antibodies comprise IgG and IgM antibodies.

11. The combination of materials of claim 10, wherein the polyclonal goat anti-feline IgG and IgM antibodies are capable of specifically binding to anti-feline and anti-canine antibodies.

12. The combination of materials of claim 11, wherein the at least one Trypanosoma cruzi antigen comprises a combination of 1F8, Tc24, SAPA, B13, PEP2, FRA, TSSA, TcD, TcF, TcE, an LA204 protein selected from the proteins in Table B, and an IBMP-8 protein selected from the proteins of Table B.

13. The combination of materials of claim 11, wherein the at least one Trypanosoma cruzi antigen comprises all of the proteins as in Table A.

14. The combination of materials of claim 11, wherein the at least one Trypanosoma cruzi antigen comprises also comprises the 1F8 or the IBMP-8 or an antigenic fragment thereof.

15. The combination of materials of claim 11, wherein the at least one Trypanosoma cruzi antigen comprises also comprises the 1F8 or the IBMP-8 protein.

16. The combination of materials of any one of claims 1-15, further comprising a liquid biological sample obtained from a feline or canine.

17. The combination of materials of any one of claims 1-15, wherein the combination of materials are provided in a kit, the kit further comprising one or more sealed containers that contain the substrate comprising the one or more proteins or antigenic fragments thereof, and one or more containers that contain the detectably labeled antibodies, the kit optionally further comprising printed material that identifies the combination of materials and/or provides instructions to a user for using the combination of materials to determine antibodies from a subject.

18. A method comprising exposing a biological sample obtained from a mammal to a material of any one of claims 1-15, and determining a signal from the detectably labeled antibodies.

19. The method of claim 18, wherein the mammal is a canine or feline animal.

20. The method of claim 18, wherein the mammal is a canine.

21. The method of claim 18, wherein the mammal is a feline.

22. The method of claim 18, wherein the signal from the detectably labeled antibodies signifies the mammal has been exposed to Trypanosoma cruzi.

23. The method of claim 18, wherein the signal from the detectably labeled antibodies aids in a determination that the mammal has Chagas disease.

24. The method of claim 23, further comprising administering to the mammal at least one therapeutic agent for treatment of the Chagas disease.

25. A method comprising exposing a biological sample obtained from a mammal to a material of any one of claims 7-15, and determining a signal from the detectably labeled antibodies.

26. The method of claim 25, wherein the mammal is a canine or feline animal.

27. The method of claim 25, wherein the mammal is a canine.

28. The method of claim 25, wherein the mammal is a feline.

29. The method of claim 25, wherein the signal from the detectably labeled antibodies signifies the mammal has been exposed to Leishmania.

30. The method of claim 24, wherein the signal from the detectably labeled antibodies aids in a determination that the mammal has Leishmaniasis.

31. The method of claim 30, further comprising administering to the mammal at least one therapeutic agent for treatment of the Leishmaniosis.

33. The method of claim 25, wherein the signal from the detectably labeled antibodies signifies the mammal has been exposed to Dirofilaria immitis.

33. The method of claim 32, wherein the signal from the detectably labeled antibodies aids in a determination that the mammal has heartworm disease.

34. The method of claim 33, further comprising administering to the mammal at least one therapeutic agent for treatment of the heartworm disease.

35. A combination of materials comprising:
a) a substrate comprising at least one Leishmania antigen attached to the substrate, wherein said antigen is optionally selected from Leishmania protein rK39, rK28, rKR95, rK18, K39, K28, KR95, and K18, and antigenic fragments thereof; and
b) detectably labeled antibodies that are capable of specifically binding to antibodies produced by an animal that has been exposed to Leishmania.

36. A combination of materials comprising:
a) a substrate comprising at least one at least Dirofilaria immitis antigen attached to the substrate, wherein said antigen is optionally selected from DIT33, ChimDiT33, PDi33, and antigenic fragments thereof; and
b) detectably labeled antibodies that are capable of specifically binding to antibodies produced by an animal that has been exposed to the Dirofilaria immitis antigen attached to the substrate.
